(19) United States
(12) Patent Application Publication
(10) Pub. No.: US 2026/0102072 A1
(43) Pub. Date: Apr. 16, 2026

(54) PORTABLE MULTI-CHANNEL SPECKLE CONTRAST OPTICAL SPECTROSCOPY SYSTEMS FOR CEREBRAL BLOOD FLOW AND BLOOD VOLUME MEASUREMENT IN BRAIN INJURY

(71) Applicants: California Institute of Technology, Pasadena, CA (US); University of Southern California, Los Angeles, CA (US)

(72) Inventors: Simon Mahler, Marseille (FR); Yu Xi Huang, South Pasadena, CA (US); Changhuei Yang, South Pasadena, CA (US); Charles Liu, San Marino, CA (US)

(73) Assignees: California Institute of Technology, Pasadena, CA (US); University of Southern California, Los Angeles, CA (US)

(21) Appl. No.: 19/374,890

(22) Filed: Oct. 30, 2025

Related U.S. Application Data
(63) Continuation-in-part of application No. 18/935,159, filed on Nov. 1, 2024.
(60) Provisional application No. 63/547,269, filed on Nov. 3, 2023.
(60) Provisional application No. 63/713,788, filed on Oct. 30, 2024.

(51) Int. Cl. A61B 5/026; A61B 5/00

(52) U.S. Cl. CPC A61B 5/0261; A61B 5/6803; A61B 5/7282

(57) ABSTRACT

Techniques for measuring cerebral blood metrics such as cerebral blood flow and/or cerebral blood volume at multiple regions of the brain and detecting a brain injury or other brain malfunction by comparing cerebral blood metrics measurements at different regions.

### CROSS-REFERENCES TO RELATED APPLICATION

This application claims priority to and benefit of U.S. Provisional Patent Application No. 63/713,788, filed on Oct. 30, 2024, and titled “Portable Multi-Channel SCOS System for Cerebral Blood Flow and Volume Measurement in Brain Injury;” this application is a continuation-in-part of U.S. patent application Ser. No. 18/935,159, filed on Nov. 11, 2024, and titled “COMPACT LASER-POWERED SPECKLE VISIBILITY SPECTROSCOPY DEVICES,” which claims priority to and benefit of U.S. Provisional Patent Application No. 63/547,269, titled “Compact and Cost-Effective Laser-Powered SVS Device for Assessing Cerebral Blood Flow and Cerebrovascular Reactivity,” filed on Nov. 3, 2023; this application is also a continuation-in-part of PCT application PCT/US24/54216, filed on Nov. 1, 2024, and titled “COMPACT LASER-POWERED SPECKLE VISIBILITY SPECTROSCOPY DEVICES,” which claims priority to and benefit of U.S. Provisional Patent Application No. 63/547,269; all of these applications are incorporated by reference herein in their entireties and for all purposes.

### FEDERALLY SPONSORED RESEARCH OR DEVELOPMENT

This invention was made with government support under Grant No. EY033086 awarded by the National Institutes of Health. The government has certain rights in the invention.

### FIELD

Certain aspects generally relate to techniques for measuring cerebral blood metrics such as cerebral blood flow and cerebral blood volume, and more specifically, to methods and systems for determining a likelihood of brain injury from cerebral blood flow metrics.

### BACKGROUND

Brain injury can occur from traumatic and non-traumatic mechanisms. Traumatic brain injury (TBI) is one of the leading causes of death and disability among young people worldwide. A TBI occurs when the brain experiences excessive non-physiological mechanical forces which can lead to hemorrhage, contusion, inflammation, cell death, edema, and/or ischemia. After recovery from TBI, patients can exhibit persistent evidence of structural brain damage as seen on MRI, as well as physiological disruptions such as cerebrovascular dysregulation that lead to subtle functional deficits that may worsen with time if left untreated. While structural sequela of TBI can be readily characterized by MRI, objective measures of cerebrovascular reactivity and cerebral blood flow can be very helpful in fully characterizing the effect of TBI, including mild cases not associated with obvious structural damage to the brain. The incidence of mild TBI with minimal structural brain damage is particularly high—there are nearly three million mild TBI occurrences in the US each year with the majority occurring in adolescents and young adults. Mild TBI is also one of the leading causes of injuries in the U.S. Army, with blast-related TBI often described as the signature injury during deployment. Despite the heavy injury toll and almost two decades of research, the diagnosis, treatment, and recovery from TBI remains poorly understood. Some techniques have been used to characterize TBI such as MRI-based neuroimaging, electrophysiology, blood and saliva biomarkers. Beyond TBI, non-traumatic brain injury (NTBI) can also lead to structural and physiological sequela including conditions such as stroke, hypoxia, infections, and toxic exposures. These injuries can result in damage to brain tissue, leading to long-term cognitive, motor, and sensory deficits depending on the affected brain regions.

### SUMMARY

Techniques disclosed herein may be practiced with a processor-implemented method, systems comprising one or more processors and one or more processor-readable media, and/or one or more non-transitory processor-readable media.

Certain embodiments pertain to headsets or other systems for generating cerebral blood metric data. In some embodiments, a headset for generating cerebral blood metric data includes a headband configured to be worn on a head, a laser coupled to the headband and configured to emit light into a brain within a skull of the head, a light detector coupled to the headband and configured to generate information indicative of light reflected from one or more structures within the brain, wherein the light detector is configured to contact or be within 5 mm of a scalp of the skull, and a power source coupled to the headband and in electrical communication with the laser.

In some embodiments, a headset may be free of optical fibers.

In some embodiments, a speckle contrast optical spectroscopy (SCOS) system has a low profile.

In some embodiments, the headset is a multi-channel headset including a headband configured to be worn on a head during operation; and a plurality of channels coupled to the headband. Each includes a laser configured to emit light into a brain within a skull of the head, a light detector configured to generate information indicative of light emitted by the laser and reflected by one or more structures within the brain, and a power source in electrical communication with the laser. The light detectors of the channels are configured to probe different regions of the brain.

In some embodiments, SCOS systems include a circuit board, one or more processors attached to the circuit board, a laser diode attached to the circuit board, a light detector in electrical communication with the one or more processors (e.g. an application-specific integrated circuit or a field programmable gate array), and a light block located between the laser diode and the light detector (e.g., one or more CMOS sensors). A power source may be in electrical communication with the laser diode. In some cases, SCOS systems also include a wireless transmitter. One or more of these SCOS systems may be attached to a headband according to an implementation.

Certain embodiments pertain to methods for generating cerebral blood metric data. In some embodiments, a method includes causing, using a laser, light to be emitted into a brain within a skull of a head of a user, obtaining, using a light detector, information indicative of light reflected from one more structures within the brain, and based on the obtained information, determining one or more cerebral blood metrics as a function of time. In some cases, the method may also include normalizing each speckle image based on a first set of the speckle images acquired during a first time period, calculating a speckle contrast of each normalized speckle image, and adjusting the speckle contrast to account for noise.

Certain embodiments pertain to multi-channel speckle contrast optical spectroscopy systems. In some cases, the systems include a headset with a headband configured to encircle a head of a subject; one or more light sources attached to the headband; a plurality of light detectors attached to the headband, the plurality of light detectors corresponding to a plurality of channels; and one or more processors. The one or more processors are configured to: cause, using one or more light sources disposed on a headset worn by a subject, light to be emitted into a brain of the subject; simultaneously obtain, using light detectors of a plurality of channels disposed on the headset, information indicative of light reflected from one more structures in a plurality of corresponding regions within the brain; based on the obtained information, determine one or more cerebral blood metrics for the plurality of channels; determine two-channel correlation factors for channel pairs of the plurality of channels based on the one or more cerebral blood metrics determined; and determine the likelihood of brain malfunction at a region proximate one of the channels based on part on a determination that one or more of the two-channel correlation factors associated with the one of the channels is less than a first threshold.

Certain embodiments pertain to speckle contrast optical spectroscopy (SCOS) methods. In some embodiments, a SCOS method includes: causing, using one or more light sources disposed on a headset worn by a subject, light to be emitted into a brain of the subject; simultaneously obtaining, using light detectors of a plurality of channels disposed on the headset, information indicative of light reflected from one more structures in a plurality of corresponding regions within the brain; based on the obtained information, determining one or more cerebral blood metrics for the plurality of channels; determining two-channel correlation factors for channel pairs of the plurality of channels based on the one or more cerebral blood metrics determined; and determining the likelihood of brain malfunction at a region proximate one of the channels based on part on a determination that one or more of the two-channel correlation factors associated with the one of the channels is less than a first threshold. In some embodiments, a SCOS method for determining a likelihood of a brain malfunction includes: causing, using one or more light sources disposed on a headset worn by a subject, light to be emitted into a brain of the subject; obtaining, using one or more light detectors disposed on the headset, information indicative of light reflected from one more structures in a plurality of corresponding regions within the brain; based on the obtained information, determining one or more time traces of a cerebral blood metric at one or more corresponding regions of the brain; comparing morphological features of the one or more time traces with a reference time trace; and predicting the brain malfunction at a region of the brain proximal one or more light detectors based in part on the comparison.

These and other features are described in more detail below with reference to the associated drawings.

### BRIEF DESCRIPTION OF THE DRAWINGS

Certain aspects are illustrated by way of example and not limitation in the figures of the accompanying drawings in which like references indicate similar elements.

FIG. 1A depicts a schematic illustration of components of a SCOS system for generating cerebral blood metrics data, according to some embodiments.

FIG. 1B depicts a schematic illustration of an example of components of a SCOS system for generating cerebral blood metrics data, according to some embodiments.

FIG. 2 depicts a schematic illustration of a top view of the SCOS system in FIG. 1A illustrating example light paths through the skull and a portion of a brain of a user, according to some embodiments.

FIG. 3A depicts a drawing of an isometric view of a light emission package, according to some embodiments.

FIG. 3B depicts a drawing of an exploded view of the light emission package in FIG. 3A.

FIG. 4A depicts a drawing of an isometric view of a light detector package, according to some embodiments.

FIG. 4B depicts a drawing of an exploded view of the light detector package in FIG. 4A.

FIG. 5A depicts a drawing of an isometric view of a light detector package having a heat sink, according to some embodiments.

FIG. 5B depicts a drawing of the heat sink in FIG. 4A.

FIG. 6 depicts a drawing of an exploded view of the light detector package in FIG. 5A.

FIG. 7 depicts a block diagram of a SCOS system with a headset having one or more channels, according to various embodiments.

FIG. 8A depicts a schematic illustration of an example of a multi-channel SCOS system for generating cerebral blood metrics data, according to some embodiments.

FIG. 8B depicts a schematic illustration of a top view of components of the multi-channel SCOS system in FIG. 8A, according to some embodiments.

FIG. 9A depicts a schematic illustration of the computing system of the multi-channel SCOS system in FIG. 8A, according to an implementation.

FIG. 9B depicts plots of traces of cerebral blow flow and cerebral blood volume metrics measured over time by the multi-channel SCOS system in FIG. 8A, according to some embodiments.

FIG. 10A depicts a schematic illustration of an isometric view of an SCOS system for generating cerebral blood metrics data, according to some embodiments.

FIG. 10B depicts a schematic illustration of a top view of the SCOS system in FIG. 10A, according to some embodiments.

FIG. 10C depicts a schematic illustration of a side view of the SCOS system in FIG. 10A, according to some embodiments.

FIG. 11A depicts a schematic illustration of an isometric view of a multi-channel SCOS system for generating cerebral blood metrics data with a segmented light detector, according to some embodiments.

FIG. 11B depicts a schematic illustration of a top view of the SCOS system in FIG. 11A, according to some embodiments.

FIG. 11C depicts a schematic illustration of a side view of the SCOS system in FIG. 11A, according to some embodiments.

FIG. 12 depicts a schematic illustration of an example of a process flow for determining cerebral blood flow from raw images, according to embodiments.

FIG. 13 depicts a schematic illustration of an example of a process flow for determining cerebral blood flow from raw images, according to embodiments.

FIG. 14 depicts a flowchart of an example method of determining cerebral blood metrics, according to embodiments.

FIG. 15 depicts images illustrating a normalization operation of a method of determining cerebral blood metrics, according to embodiments.

FIG. 16 depicts example graphs of cerebral blood metric data based on different source-to-detector (S-D) distances for a subject, in accordance with embodiments.

FIG. 17 depicts example graphs of cerebral blood metric data based on different source-to-detector (S-D) distances for ten subjects, in accordance with embodiments.

FIG. 18 depicts example graphs of cerebral blood flow during a breath hold task in accordance with some embodiments.

FIG. 19 depicts example graphs of cerebral blood flow during a breath holding task in accordance with some embodiments.

FIG. 20 depicts example graphs of cerebral blood flow and cerebral blood volume during a breath holding task in accordance with some embodiments.

FIG. 21A is a plot of the laser optical power over time for three different power sources in accordance with some embodiments.

FIG. 21B is a plot of the average contact temperature measured for three types of cameras in accordance with some embodiments.

FIG. 22A is a plot of the contact temperature measurements over time of five healthy subjects in accordance with some embodiments.

FIG. 22B is a plot of subject comfort ratings over time for five healthy subjects in accordance with some embodiments.

FIG. 23A includes a plot of the CBF time traces of six regions in the brain of a healthy subject in accordance with some embodiments.

FIG. 23B includes a plot of CBF time traces of six different regions in the brain of a traumatic brain injury (TBI) subject in accordance with some embodiments.

FIG. 23C includes a plot of CBF time traces at six different regions in the brain of a non-traumatic brain injury (NTBI) subject in accordance with some embodiments.

FIG. 24A is a plot of the mean intensity of recordings at six channels for the five healthy subjects, the TBI subject, and the NTBI subject in accordance with some embodiments.

FIG. 24B is a plot of the two-channel correlation factors from recordings at the six channels for the five healthy subjects, the TBI subject, and the NTBI subject in accordance with some embodiments.

FIG. 25A depicts a correlation matrix with correlation factors for channel pairs of all six channels determined for a healthy subject based on the CBF time traces shown in FIG. 23A in accordance with some embodiments.

FIG. 25B depicts a correlation matrix with correlation factors for channel pairs of all six channels determined for an NTBI subject based on the CBF time traces shown in FIG. 23C in accordance with some embodiments.

FIG. 26 is a plot of the two-channel correlation factor as a function of the two-channel mean intensity for all subjects.

FIG. 26 is a plot of the two-channel correlation factors and two-channel mean intensities in accordance with some embodiments.

FIG. 27 is a flow diagram depicting a method of determining a likelihood of a brain malfunction such as a brain injury, according to some embodiments.

FIG. 28A is a plot of an example of one reference pressure waveform of a time trace of CBFI in a healthy subject in accordance with some embodiments.

FIG. 28B is a plot of an example of a pressure waveform of a time trace of CBFI measured in the NTBI subject at channel 1 proximate the brain injury in accordance with some embodiments.

FIG. 29 is a flow diagram depicting a method of determining a likelihood of a brain malfunction such as a brain injury, according to some embodiments.

FIG. 30 depicts a diagram of components of an example computing device in accordance with some embodiments.

These and other features are described in more detail below with reference to the associated drawings.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

### I. Introduction

Tracking cerebral blood flow non-invasively is essential but challenging when evaluating cerebral autoregulation and detecting cerebrovascular irregularities. While ultrasound and magnetic resonance imaging (MRI) have been used to measure cerebral blood flow, optical methods may be advantageous due to their high sensitivity and temporal resolution. For example, optical techniques are more sensitive than ultrasound methods since the wavelength of light being used is at least an order of magnitude smaller than the typical ultrasound wavelength. In addition, optical techniques may provide higher temporal resolution (e.g., 50-100 Hz) than MRI methods.

Speckle contrast optical spectroscopy (SCOS) (also referred to as speckle visibility spectroscopy (SVS)) is a technique that can be used to infer blood flow from the spatial ensemble of speckle fields in images captured by a camera. In SCOS, tissues being probed are illuminated and photons reflected from structures in the tissues are collected by pixels of the camera within the same frame. The camera typically acquires images with an exposure time longer than the decorrelation time of the speckle field. This results in multiple different speckle patterns being summed up into a single camera frame. As the speckle field fluctuates, each speckle pattern recorded by the camera is smeared and washed out within the exposure time. The smearing or washing out is due to the dynamics of the blood cells. A decorrelation time can be calculated from the degree of blurring of the captured frame, typically by calculating the speckle contrast. Blood flow may be determined from the speckle contrast.

Described herein are techniques that use SCOS to determine cerebral blood metrics. In particular, certain techniques pertain to systems that use SCOS to monitor cerebral blood metrics such as cerebral blood volume (CBV), cerebral blood flow (CBF), and/or heart rate. These SCOS systems that use SCOS are sometimes referred to herein as SCOS apparatuses or SCOS devices. In some cases, the data used to determine cerebral blood metrics may be obtained during a period of time that includes time a user such as a patient is holding their breath (sometimes referred to herein as a “breath-holding time period”) or a period of time before or after the breath-holding time period. For example, normalization data may be obtained during a baseline time period before the breath-holding time period.

Cerebral blood metrics may be determined using data characterizing light reflected from various structures within a brain of the user. For example, in some implementations, cerebral blood volume, cerebral blood flow and/or heart rate metrics may be determined by transmitting light in a near-infrared or infrared wavelength into the brain of the user and measuring reflected light. In some implementations, cerebral blood flow may be determined from the speckle contrast in images based on the reflected light from the brain using SCOS.

In certain implementations, a SCOS system includes a laser (e.g. laser diode) or other light source and a light detector, which may be a camera, or the like. The light detector may include one or more sensors (e.g., one or more CMOS sensors). The light source and light detector may be attached to a headband or other structure that can encircle the head of a user such as, e.g., around the forehead. Light may be transmitted through the skull into the brain using the light source. Reflected light from one or more structures in the brain may be captured using the light detector. The light detector is configured in the headband to be in close proximity to, or in direct contact with, the scalp when the headband is encircling the head. In some cases, the headband may be tightened to apply pressure to the scalp via contact with the light detector to reduce blood flow locally. Placing the light detector as close as possible to skull maximizes the numerical aperture of the optical imaging system and number of photons received by the light detector. This configuration is also advantageous over optical systems with collection optical fiber between the scalp and the light detectors. For example, optical systems relying on fiber collection can be susceptible to fluctuations caused by the movement of collection fiber which affects speckle dynamics. By placing the light detector in direct contact with the scalp surface, this source of instability is avoided, and more consistent results are achieved with a simpler arrangement. In one example, the SCOS system can achieve a sampling rate of 50 Hz while being minimally affected by external disturbances.

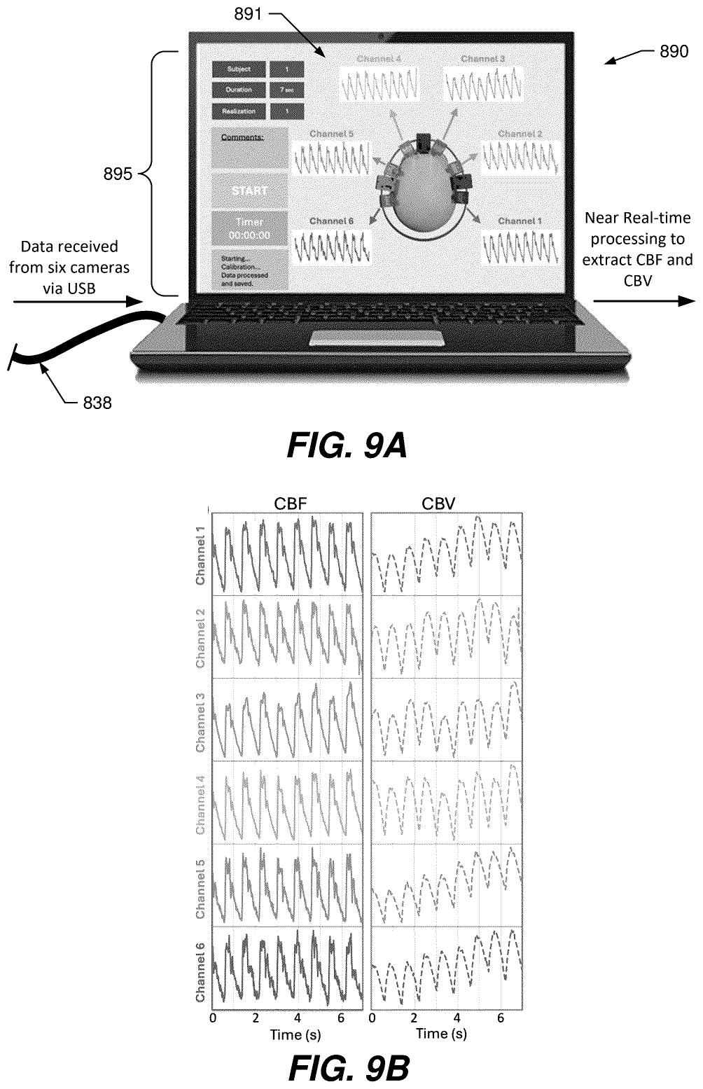

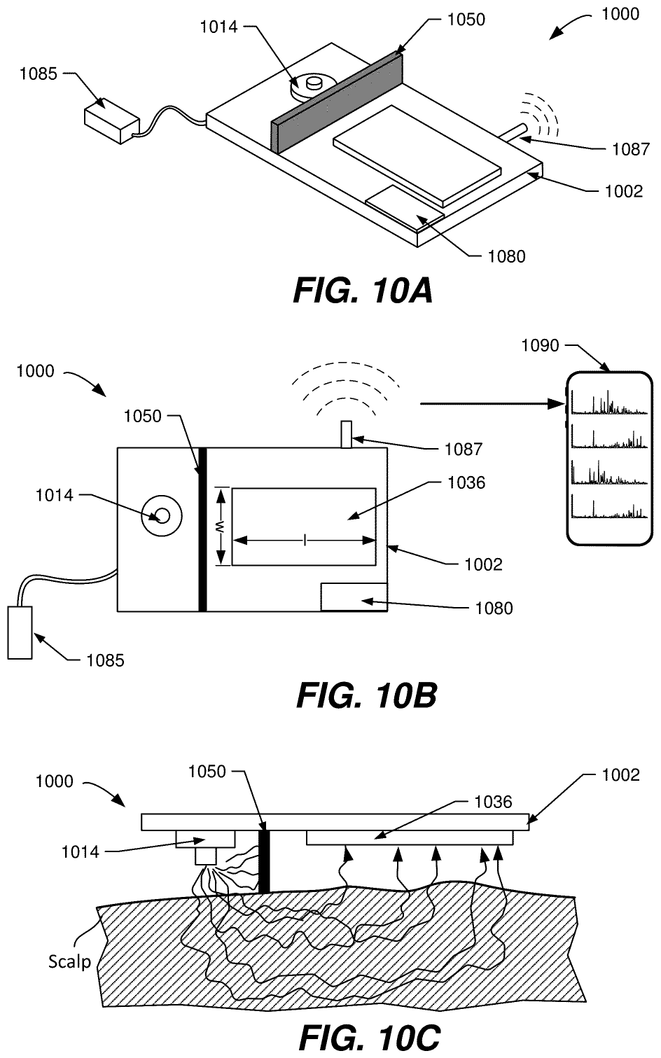

Certain implementations pertain to compact SCOS systems for non-invasively monitoring cerebral blood flow (CBF) and/or cerebral blood volume (CBV), and cerebrovascular reactivity. In some cases, the compact system includes a light source such a laser diode and a light detector such as a CMOS board camera that are in a single package that can be placed on a head to measure CBF/CBV. An example of components of a compact SCOS system are shown in FIGS. 9A-9C. Another example of components of a compact SCOS system are shown in FIGS. 10A-10C. In some instances, these SCOS systems may provide real-time CBF/CBV monitoring at, for example, a 50 Hz sampling rate. These SCOS systems may also provide the advantages of being lightweight and low cost.

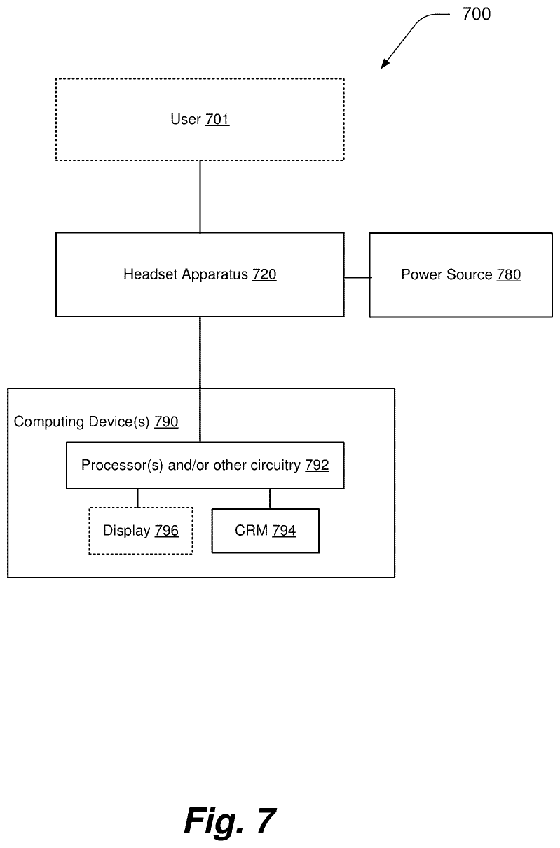

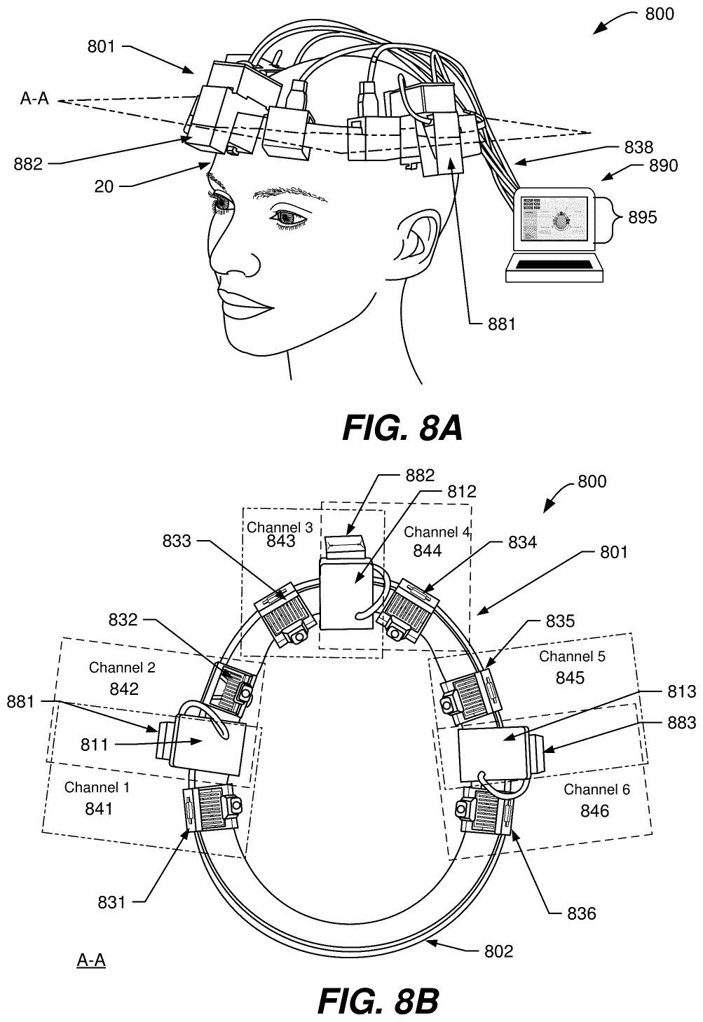

In some embodiments, a SCOS system includes multiple light sources and multiple light detectors. For example, a SCOS system may be a multi-channel SCOS system with multiple light source and light detector (source-detector) pairs forming respective channels such as the multi-channel SCOS system 810 with six channels 841, 842, 843, 844, 845, and 846 shown in FIGS. 7A-B. Generally speaking, a multi-channel SCOS system may have any suitable number of channels (e.g., 2, four, six, eight, ten, etc.) disposed at various locations to be able to probe different regions of the brain simultaneously or at different times. In some instances, two or more channels may share the same light source. For example, as shown in FIG. 8B, channel 1 841 shares a light source 811 with channel 2 842.

In some embodiments, one or more light sources (sometimes referred to herein as “sources”) are packaged as a light emission package and a light detector (sometimes referred to herein as a “detector”) is packaged as a light detector package. In some implementations, one or more light emission packages and one or more light detector packages may be coupled to a headband or cap at different positions or may be adjustable to move along the headband or cap to different positions to be able to take simultaneous measurements of different regions of the brain such the parietal lobe, the frontal lobe, etc. A light source may be coupled to the headband or cap via the light emission package and a light detector may be coupled to the headband or cap via the light detector package.

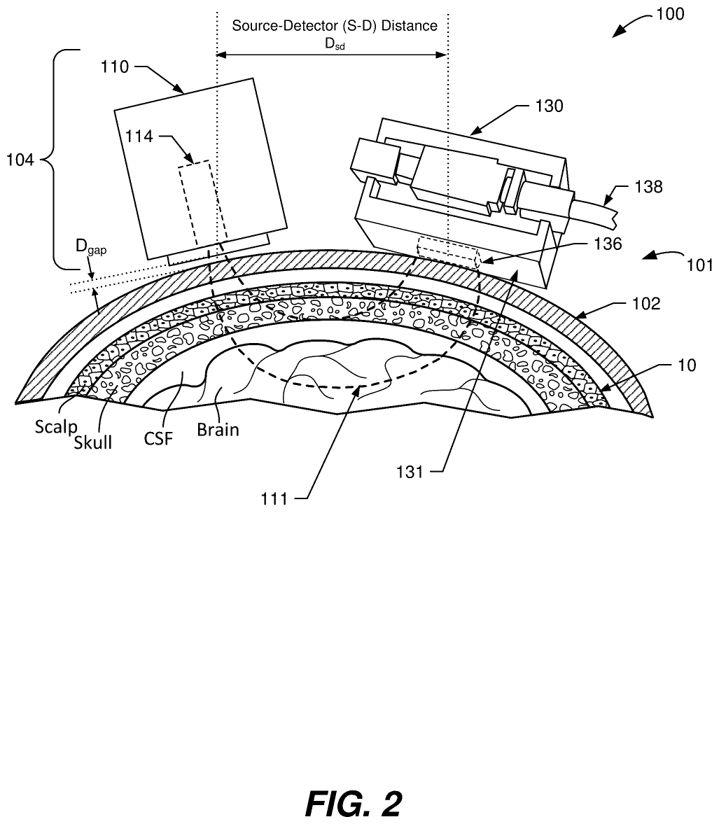

The distance between the illumination spot of the light source and a corresponding light detector is generally referred to as the “source-detector distance,” or “S-D distance” and example values may include 2 cm, 2.5 cm, 3 cm, 3.5 cm, 4 cm, etc. In some implementations, the S-D distance is within a range of about 3.0 cm-4 cm. In some implementations, the S-D distance is within a range of about 2.5 cm-3.5 cm. In some implementations, the S-D distance is within a range of about 3.0 cm-5.0 cm. An S-D distance of between 3.0 cm and 4.0 cm may provide the advantage that the majority of collected photons have traveled through portions of the brain which may provide the high brain specificity needed to detect cerebral blood flow. In instances in which a headset includes multiple channels, the distance between the light detector and the corresponding light source for different channels may be different. In some implementations, positions of light sources and/or light detectors may be modifiable. For example, a light emission package and/or a light detector package may be adjustable in position via screws, Velcro, or other similar adjustable hardware. This may allow for S-D distances to be modified, which may in turn allow the depth of imaging to be modified as different source-detector distances impact the depth the emitted light can penetrate within the brain, as shown in and described below in connection with FIG. 2. Additionally, modification of positions of light sources and/or light detectors may allow different brain regions to be probed simultaneously using one headset.

In some embodiments, a SCOS system may have a light source and light detector coupled to a single circuit board, which is sometimes referred to herein as a combined source-detector package. In some cases, this SCOS system includes one or more processing units or processors (e.g., an application-specific integrated circuit (ASIC) or a programmable logic device such as a field-programmable gate array (FPGA)), a laser diode, and a light detector coupled to the circuit board. The SCOS system also includes a light block (e.g., a wall of opaque material) located between the laser diode and the light detector to avoid cross talk. The SCOS system also includes a power source in electrical communication with the laser diode. An example of such an SCOS system is SCOS system 1000 shown in FIGS. 10A-10C, which is described in more detail below. In some cases, the light detector may be segmented. For example, the light detector may include multiple sensors arranged at different locations at different S-D distances from the laser diode to probe different regions. In these cases, the SCOS system includes multiple channels corresponding to the light source and different detector segments.

In some implementations, the light detector package may be fiber free in that there is no optical fiber coupling to the light detector. In these cases, the light detector may be placed directly atop the user's scalp. In addition or alternatively, the light emission package may be fiber free since the light source is an integral part of the light emission package and does not required optical fiber to communicate the light from an external source. In various embodiments, all components of a headset may be fiber free, which avoids noise emanating from optical fibers.

In addition to measuring cerebral blood flow, the SCOS system of various embodiments can measure cerebral blood volume and heart rate, allowing the simultaneous measurement of multiple cerebral blood metrics at single or multiple regions of the brain. With its cost-effectiveness, scalability, versatility, and user-friendliness, the laser-based apparatus of various examples hold great promise for advancing non-invasive cerebral monitoring technologies.

In some embodiments, a light detector may be divided into segments where the segments can simultaneously generate data based on light collected from different regions of the brain. Segmentation allows data for different depths (corresponding to different source-detector distances) to be measured simultaneously using a single light detector. Segmentation may also help calibrate noise and provide redundancy by effectively using one light detector as multiple image generating sources. For example, the light detector may be composed of multiple sensors such as multiple CMOS sensors at different locations that can capture frames from different regions of the brain simultaneously. In some cases, the light detector may include multiple CMOS sensors arranged at different S-D distances from the light source along a length to capture frames of regions at different depths within the brain. As another example, the light detector may include one sensor with a two-dimensional array of pixels where the array is divided into different sets (segments) of pixels at different locations.

In cerebrovascular monitoring, primary metrics typically include blood pressure, which influences cerebral blood flow, and the cerebral blood volume, which is contingent upon vessel radius. In typical cerebral autoregulation situations, the autoregulation system attempts to maintain a constant blood pressure by modulating the blood vessel's diameter, which is measured by blood volume. However, the extent to which blood vessels can be dilated is limited and there exist some breakdown regime where vessels can no longer dilate to modulate the increasing blood pressure. In some implementations, the SCOS system or a separate computing device may detect a breakdown zone from the cerebral blood metrics. Ideally, when the breakdown zone is detected, further physiological tests will be conducted.

Certain embodiments pertain to SCOS systems (e.g. SCOS system 700 in FIG. 7, multi-channel SCOS system 800 in FIGS. 8A-8B, etc.) that can perform non-invasive measurement of cerebral blood flow (CBF) and/or cerebral blood volume (CBV) in individuals with brain injuries, including both traumatic and non-traumatic instances. In certain implementations, the techniques for measuring cerebral blood flow metrics can assess the likelihood of brain injury in a subject based on an evaluation of blood flow metrics being monitored. In various implementations, multi-channel SCOS systems can provide rapid, accurate assessments of cerebral hemodynamics at different locations of the brain non-invasively, which can facilitate timely diagnosis and treatment of brain injuries.

Certain techniques for monitoring cerebral blood flow metrics described herein may advantageously provide effective, near real-time monitoring of cerebral blood dynamics in patients with brain injuries. Other diagnostic methods, such as MRI and CT scans, while effective, can often be time-consuming, expensive, and not be readily available in emergency settings. In contrast, the techniques for monitoring cerebral blood flow metrics described herein may provide a portable, cost-effective solution that can be deployed in various clinical environments, including field hospitals and ambulances. These techniques for monitoring cerebral blood flow metrics enable healthcare professionals to assess the physiological state of a patient's brain swiftly, helping to identify potential complications and guide treatment decisions.

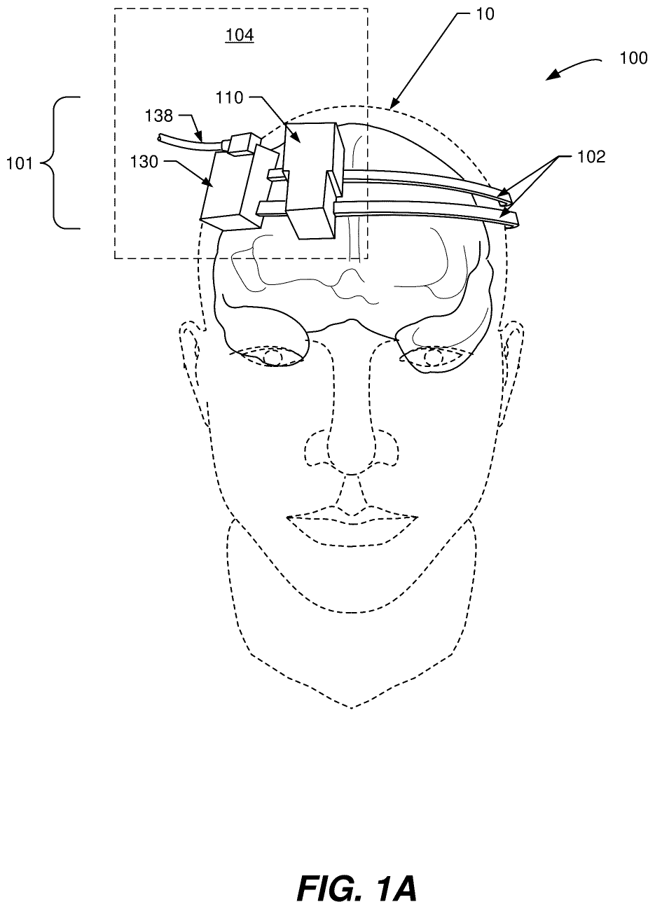

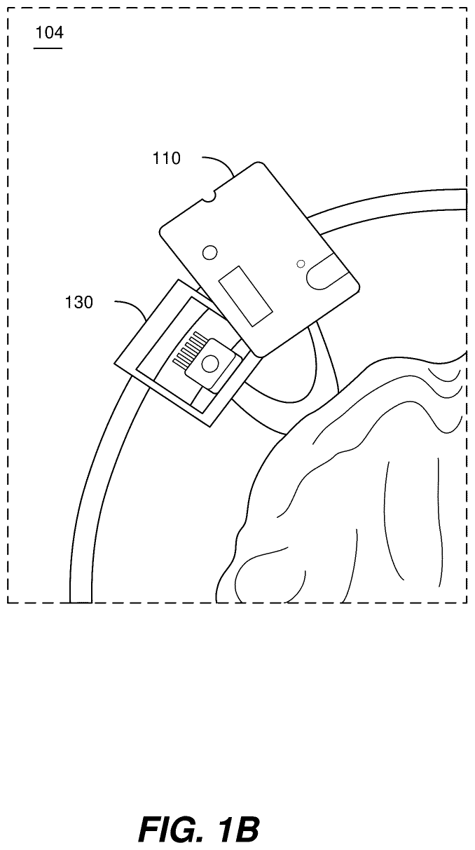

The techniques for measuring cerebral blood flow metrics of various implementations may provide one or more technical advantages. For example, certain multi-channel SCOS systems may provide portability and flexibility. For instance, unlike traditional imaging techniques (e.g., MRI) that require significant infrastructure and patient transport, the multi-channel SCOS systems can have features designed for ease of use and mobility. Multi-channel SCOS systems can be, for example, lightweight, battery-powered, and with components integrated into a wearable headset (e.g., headset 101 in FIG. 1) with a headband, making it suitable for use in diverse settings. As another example, multi-channel SCOS systems can be configured for multi-channel monitoring. For instance, multi-channel SCOS systems of various implementations include a multiple channel setup (e.g., headset 800 in FIG. 8A with a six-channel setup) that can enable measurements of CBF and CBV at multiple locations within the brain, either simultaneously or at different times. This capability enhances the ability to identify regional differences in blood flow dynamics by comparing measurements taken at different regions, which can be used to diagnose the extent of the brain injury. For instance, the measured regional differences in blood flow dynamics may be used to assess whether brain injury in a region of the brain is a non-traumatic brain injury or a traumatic brain injury according to certain implementations.

As another example, certain techniques for measuring cerebral blood flow metrics described herein can provide near real-time data processing. For instance, certain systems may have real-time data processing capabilities on a computing device (e.g., a laptop or integrated into a headset) implemented via user-friendly graphical user interface (GUI) software. This allows clinicians to visualize blood flow dynamics immediately, leading to quicker decision-making. In addition, real-time processing of raw speckle image data may be performed to extract cerebral blood metrics without the need to store the raw speckle images, which reduces computing resources needed.

As another example, certain techniques for measuring cerebral blood flow metrics described herein are non-invasive processes. For instance, certain techniques for measuring cerebral blood flow metrics use speckle contrast optical spectroscopy (SCOS) to monitor cerebral blood flow metrics (e.g., cerebral blood flow, cerebral blood volume, and/or heart rate) of a patient with a non-invasive procedure such as may be implemented by an SCOS system with a headset that can be worn on a head (outside the skull) of a patient. Non-invasive monitoring of cerebral blood flow metrics can be of particular importance in acute care scenarios.

As another example, certain techniques for measuring cerebral blood flow metrics described herein may be cost-effective. For instance, SCOS systems for measuring cerebral blood flow metrics can include low-cost optical components eliminating the need for expensive imaging equipment. These techniques may be an affordable alternative for hospitals and clinics, particularly in resource-limited settings.

Although various examples of systems described herein are implemented to monitor cerebral perfusion metrics, such as cerebral blood flow and cerebral blood volume, to evaluate brain injuries, these systems could also be used to evaluate other conditions requiring assessment of cerebral perfusion such as stroke, Alzheimer's, headaches and migraines, neurological disorders, or other cerebrovascular diseases.

In one embodiment, a SCOS system may integrate techniques for measuring cerebral blood flow metrics with other patient monitoring systems to provide additional physiological assessments, such as heart rate and blood pressure, to enhance patient care. For example, a SCOS system may include a headset for measuring cerebral blood metrics, a blood pressure monitoring system, and other monitoring systems.

In certain implementations, techniques for measuring cerebral blood flow metrics can be used as a valuable tool for research in neurology and physiology, allowing for detailed studies on blood flow dynamics in various populations, including healthy individuals, patients with chronic conditions, or during surgical procedures.

In some embodiments, a SCOS system for measuring cerebral blood flow metrics may have components in a modular design format with adjustable channels that enable customizable variations in the number of channels or the arrangement of adjustability of sensor placement. For example, a SCOS system in a modular format may include a headset that can be adjusted to add or remove one or more channels and/or move one or more channels to different locations.

### II. Examples of SCOS Systems for Generating Cerebral Blood Metrics Data

FIGS. 1A and 1B depict schematic illustrations of an example of components of a SCOS system 100 for generating cerebral blood metrics data using SCOS, according to various embodiments. FIG. 2 is a schematic diagram of a top view of the SCOS system 100 shown in FIG. 1A. In the illustrated example, the SCOS system 100 includes a headset 101 with a headband 102. The headset 101 includes a light emission package 110 having a light source 114 such as a laser and a light detector package 130 having a light detector 136 (e.g., a CMOS board camera). As shown in FIG. 1B, the light emission package 110 and light detector package 130 form a channel 104. The light emission package 110 and light detector package 130 are coupled to a portion of the headset 101. In one implementation, the light emission package 110 and light detector package 130 are adjustable to different positions along the headband 102. As shown, the headband 102 is configured to encircle at least a portion of a head 10 of a user at, e.g., the forehead level.

The SCOS system 100 also includes an electrical connection 138 (e.g., USB cable) between the light detector 136 of the light detector package 130 and one or more processors and/or a computing device (e.g., computing device 890 in FIG. 8A) for communicating data. In other implementations, SCOS system 100 may omit electrical connection 138 and includes a wireless transmitter for communicating data to a wireless receiver in electrical communication with the one or more processors and/or computer readable medium. In yet other implementations, the computing device may be integrated into the SCOS system 100 and the electrical connection 138 may be omitted. The processor(s) or computing device may perform operations to determine one or more cerebral blood metrics based on the data captured by the light detector.

In various embodiments, an SCOS system for generating cerebral blood metrics data includes a headset with a headband configured to be worn on a head of a user during operation. The headband may include a strap or a cap that can encircle the head of the user. The headband may include a tightening mechanism such as Velcro, latch, or the like that can secure and tighten the headband to the head. In some cases, the tightening of the headband may be used to place the light detector in contact with and/or apply pressure to the scalp locally where the light detector contacts the scalp. Contact pressure may be desirable to reduce blood flow at the scalp locally to decrease the scalp's influence and increase brain specificity. In addition, the headband, the one or more light detectors, and/or more or more light sources may be configured to allow adjustment of the locations of the light detector(s) and light source(s) to probe different regions/depths of the brain.

According to various embodiments, a headset may include one or more channels (e.g., one, two, three, four, five, six, seven, eight, nine, ten, etc.). For example, as shown in FIGS. 8A and 8B, the headset 901 includes six channels, 841, 842, 843, 844, 845, and 846. As another example, a headset may include four channels. By way of example, four channels may be implemented to measure cerebral blood metrics at a frontal lobe region, a parietal lobe region, or the like.

Referring back to FIG. 2, channel 104 includes a light source 114 and a light detector 136 (e.g., a camera such as a board camera). A first surface 131 of the detector package 130 is shown in contact with the scalp during operation. In some cases, the headband 102 may be tightened to apply a pressure to the scalp at a region where the detector package 130 contacts the scalp to decrease the blood flow in the scalp locally. The light source 114 may be a laser configured to emit coherent laser light in a near-infrared or infrared wavelength into the head 10 of the user. In one example, the laser may emit light at about 830 nm. In another example, the laser may emit light at about 785 nm. The light detector 136 may include one or more CMOS sensors or may be a photodiode. There is a gap between the light emission package 110 and the scalp with a gap distance, D_{gap}. In some cases, the gap distance, D_{gap}, may be in a range of 5 mm and 7 mm. In one example, the gap distance, D_{gap}, may be set to generate an illumination spot on the skull with a light intensity level that lies within safety standards.

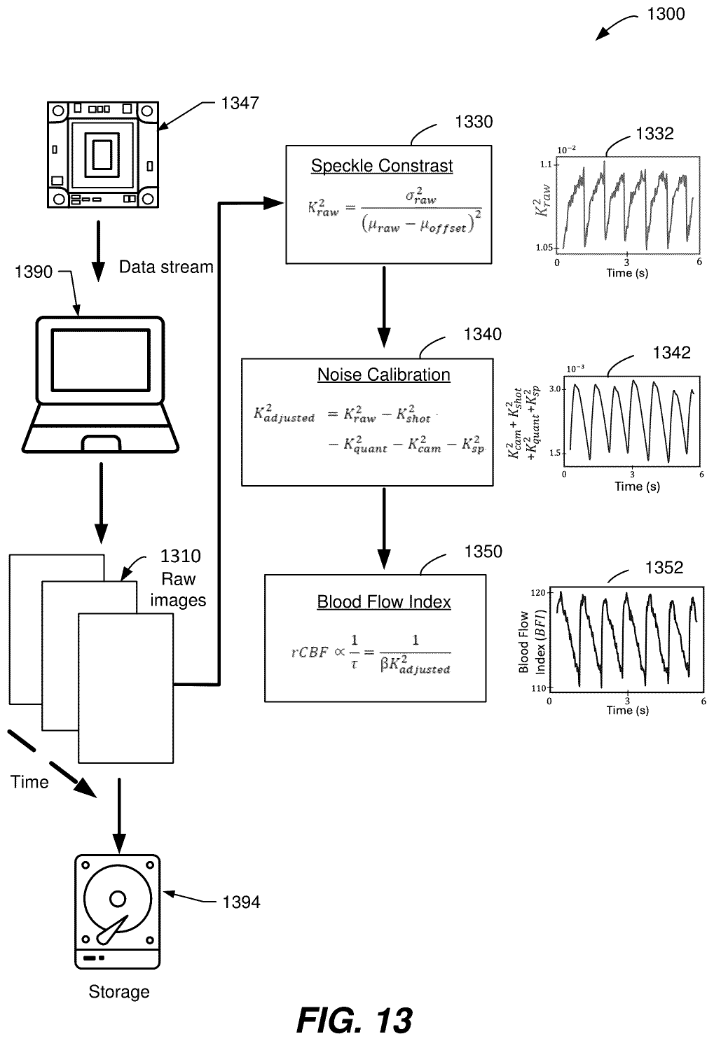

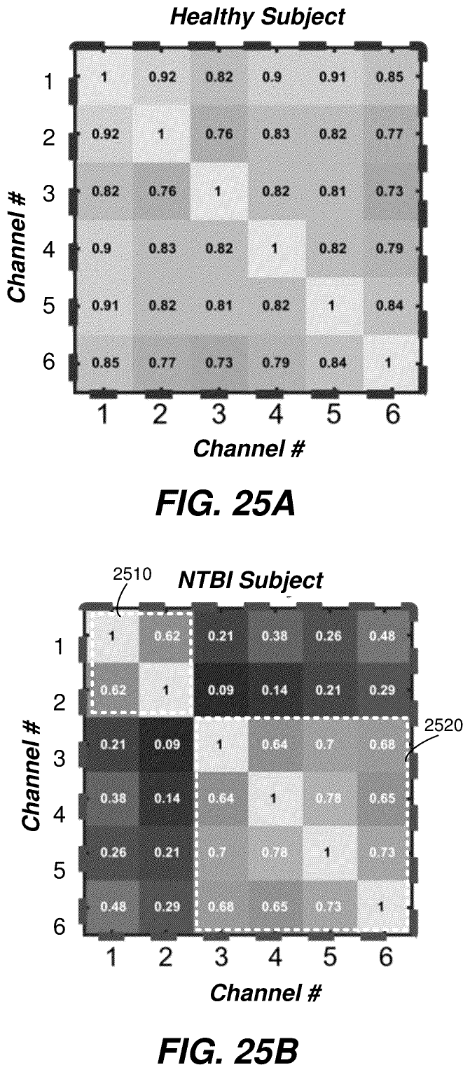

As illustrated, the light detector 136 may obtain light reflected off various brain structures from light emitted by light source 114 as illustrated by the example light paths 111. Data captured by the light detector 136 may be used to determine cerebral blood metrics. Some examples of operations that may be implemented to determine one or more cerebral blood metrics are described below in connection with FIGS. 13 and 25.

Returning to FIG. 2, the illumination spot of the light source 114 and the light detector 136 are at a source-detector distance (S-D distance), D_{sd}, from each other. The S-D distance impacts the depth that the emitted light can penetrate into the brain. In certain implementations, the S-D distance may be adjusted to tune the depth of penetration into the brain. The locations of the light detector 136 and light source 114 determine the region being imaged. In the illustration, the light detector 136 is positioned in contact with the scalp and at the S-D distance from the illumination spot of the light source 114 in order to collect light emerging from the brain at a distance away from the laser illumination spot. In some cases, the sensor area of the light detector 136 may in the range of 25 mm² and 100 mm² and the pixel pitch may be in a range of 2 μm and 4 μm. The spatial distribution of the exiting photons collected by the light detector 136 exhibits a granular pattern in the images with areas of high and low intensity, which are referred to as “speckles.” The light detector 136 is typically configured with an exposure time that is significantly larger than the decorrelation time, Tc, of the speckle field. For example, exposure times may be in a range of 1 ms and 10 ms. This results in multiple speckle patterns integrated into a single camera frame. The motions within the light paths 111 are primarily due to movement of blood cells and will scatter and change the effective optical path lengths resulting in a fluctuating speckle field that varies in time. The speckle contrast calculated from the images can be used to determine cerebral blood flow.

In various embodiments, a SCOS system for generating cerebral blood metric data includes a headset with one or more light emission packages and one or more light detector packages coupled to a headband or cap. Each light emission package includes at least one light source and each light detector package includes a light detector. In one example, the light emission package may include a plurality of light sources such as multiple laser diodes configured to emit light at different wavelengths. In other embodiments, a SCOS system includes a headset with one or more combined source-detector packages. An example of a combined source-detector package is described below in connection with FIGS. 9A, 9B, and 9C.

In various embodiments, a SCOS system for generating cerebral blood metric data includes a light source that is a laser (e.g. a laser diode). The laser may be a continuous wave laser. The laser may be configured to emit light in a near-infrared or infrared wavelength. In one example, the laser may emit light at about 830 nm. In another example, the laser may emit light at about 785 nm. In one instance, the light source may be a single-mode continuous wave 785 nm laser diode (e.g., Thorlabs laser L785H1) that can deliver up to 200 mW. In other implementations, other wavelength may be used such as 1064 nm. The light emission package may include a housing (e.g., a 3-D printed mount) within which the light source may be mounted. The light source may be placed at a predetermined distance, D_{gap}, (e.g., 6 mm) from the scalp to generate a spot diameter of 5 mm or larger during operation.

In various embodiments, a SCOS system for generating cerebral blood metric data includes a light detector, which may be a camera, or the like. A light detector may be positioned to collect light from the brain and output a data signal including data representative of one or more image frames captured over time. Each image frame may include an intensity distribution of light received at a sensing region. The light detector is configured to operate at a frame rate (e.g., 50 frames per second (fps)) and has a pixel pitch (e.g., 3.4 μm). The light detector may include one or more sensors. Some examples of suitable sensors include a complementary metal-oxide-semiconductor (CMOS) sensor, a linear or array charge-coupled device (CCD), and other similar devices. In one embodiment, the sensor may be a CMOS sensor in a flexible format such as the Cappella CMOS image sensor sold by Teledyne. In one embodiment, the light detector is a USB-Board camera (e.g., the Basler daA1920-160 μm camera with a Sony Sensor IMX392). In some cases, the light detector package may include a housing (e.g., a 3-D printed mount) within which the light detector may be mounted. The housing may be employed to prevent laser light reflections and stray light. In some cases, the light detector may include one or more CMOS sensors and have a width in a range of 5 mm and 10 mm and a length in a range of 5 mm and 10 mm. In some cases, the pixels of a CMOS sensor may have a size in a range of 2 μm and 4 μm.

According to one aspect, a light detector may be in multiple segments. This segmentation allows data from multiple source-detector distances to be measured simultaneously with a single light detector. For example, the light detector may include multiple sensors such as multiple CMOS sensors. The CMOS sensors may be arranged at different S-D distances from the light source such that the frames are captured of regions at different depths. As another example, the light detector may include one sensor with different sets (segments) of pixels at different locations that simultaneously take separate measurements.

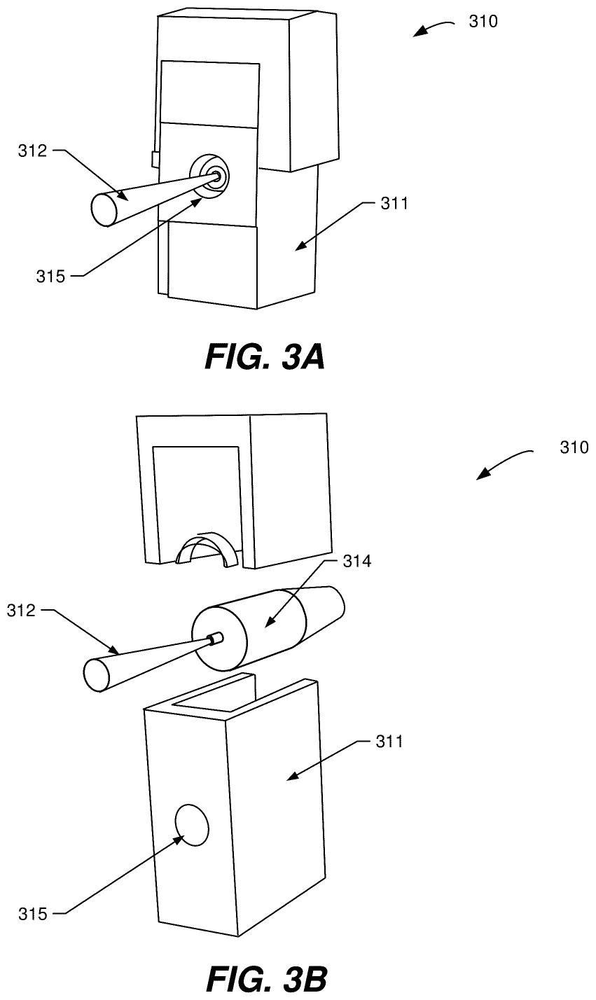

FIG. 3A is a drawing of an isometric view of an example of a light emission package 310, according to some embodiments. FIG. 3B is a drawing of an exploded view of the light emission package 310 in FIG. 3A. The light emission package 310 includes a housing 311 having an aperture 315. The light emission package 310 also includes a laser 314 mounted within the housing 311. The laser 314 is configured to emit a laser beam 312 through the aperture 315 in the housing 311. The components of light emission package 310 are examples of components that can be implemented in the light emission package 110 of SCOS system 100 in FIGS. 1A and 1B and the light emission packages of the multi-channel SCOS system 800 in FIGS. 8A and 8B.

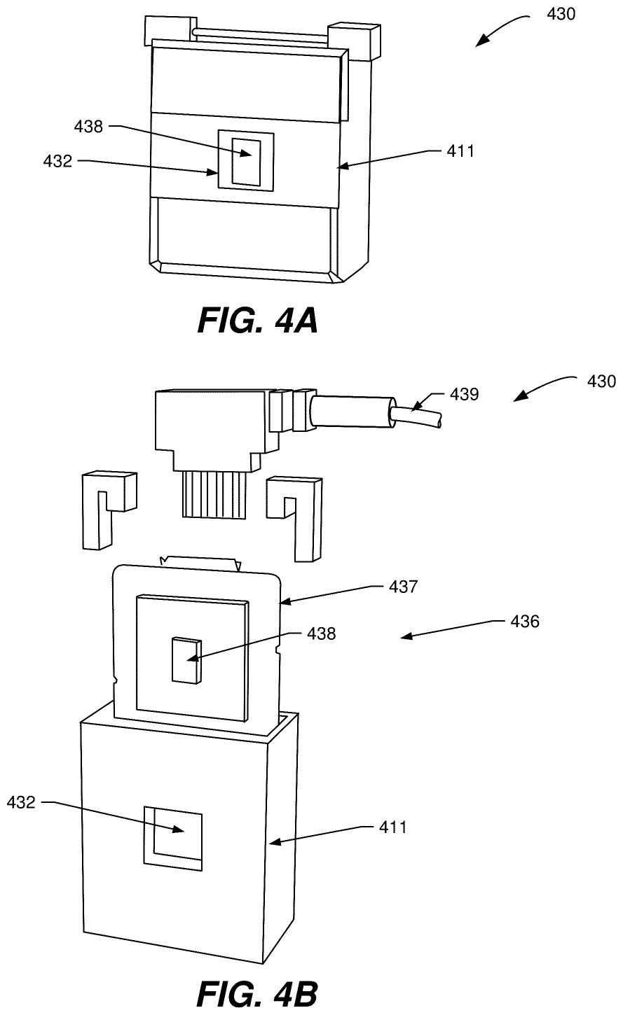

FIG. 4A is a drawing of an isometric view of a light detector package 430, according to some embodiments. FIG. 4B is a drawing of an exploded view of the light detector package 430 in FIG. 4A. The light detector package 410 includes a housing 411 having an aperture 432. The light detector package 410 also includes a light detector 436 in the form of a USB-board camera 437 (e.g., Basler daA3840-45 μm camera board) having a CMOS sensor 438 (e.g., Sony IMX392 CMOS sensor). The frame speed of the USB-board camera 437 may be about 40 fps and the number of pixels in each frame captured may be about 8.3 million pixels. The light detector 436 may be mounted to the housing 411. When assembled, the aperture 432 lies adjacent to the CMOS sensor 438 and is configured to allow light to pass to the CMOS sensor 438. The light detector package 410 also includes an electrical connection 439 that is in electrical communication with a computing device. In other implementations, a wireless transmitter may be included and the electrical connection 439 may be omitted. The components of light detector package 430 are examples of components that can be implemented in the light detector package 130 of SCOS system 100 in FIGS. 1A and 1B and the light detector packages of the multi-channel SCOS system 800 in FIGS. 8A and 8B.

In various embodiments, one or more operations of a method for determining the one or more cerebral blood metrics may be implemented by one or more computing devices or one or more processors. For example, such a computing device and/or processor may be configured to analyze data from the light detector and/or generate one or more cerebral blood metrics. In some implementations, the one or more computing device and/or one or more processors may be disposed on the same headset as the one or more light emission packages and one or more light detector packages. Additionally or alternatively, the one or more computing devices and/or one or more processors may be communicatively coupled to the headset, e.g., by a wireless or wired communication channel.

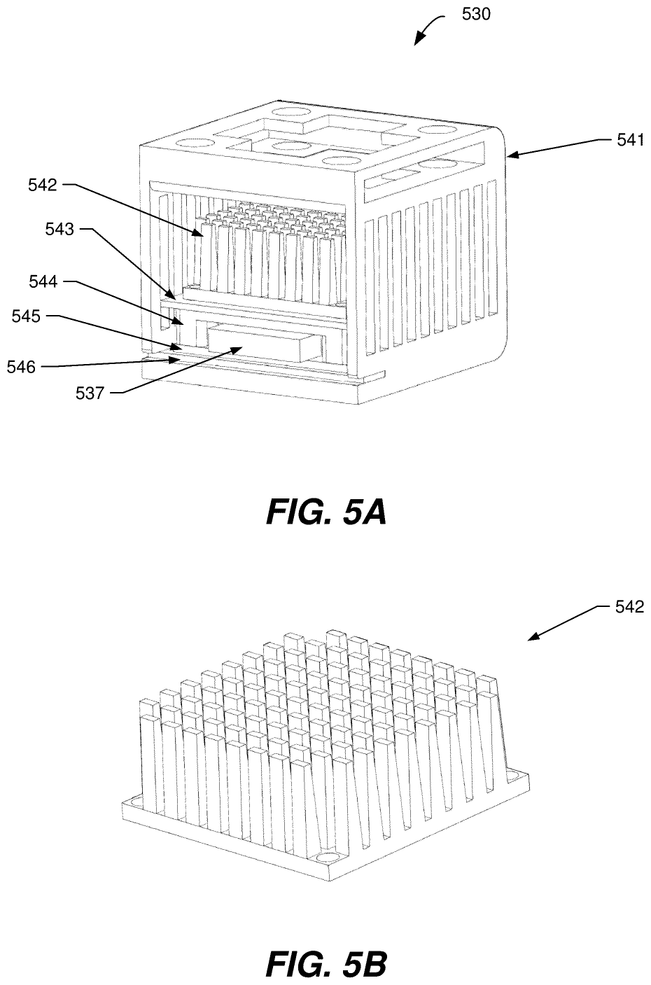

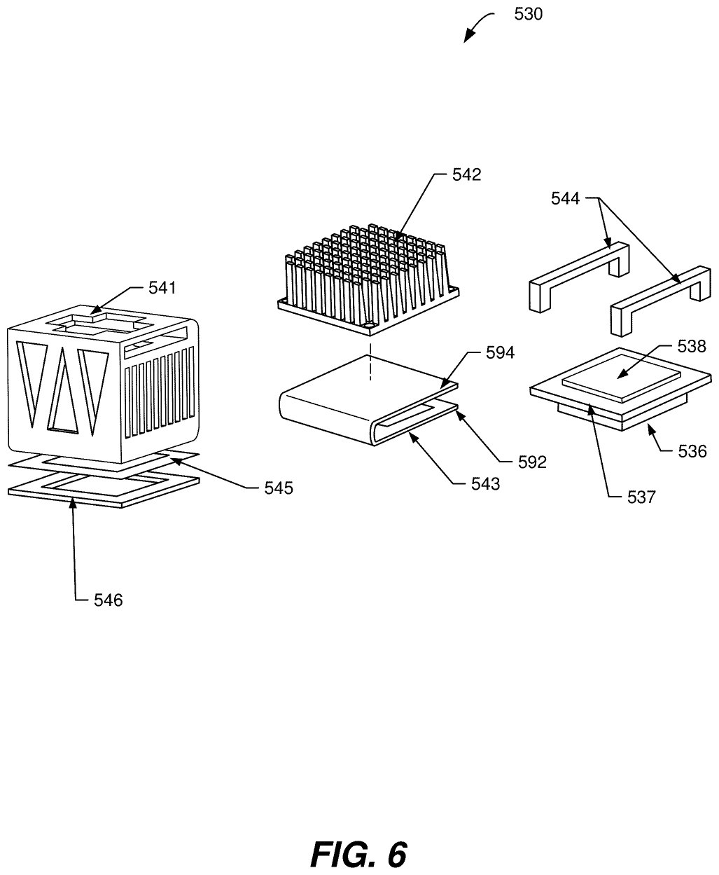

In certain implementations, the light detector package of an SCOS system includes a heat management system with one or more components designed to extract heat away from the light detector (e.g., board camera) to the external environment and/or provide insulation between the light detector and the skull of the user to reduce heat flow. For example, the heat management system may include a heat sink and one more insulative layers. FIG. 5A is a drawing of an isometric view of components of an example of a heat management system 530, according to an embodiment. FIG. 5B is a drawing of an isometric view of the heat sink 542 in FIG. 4A. FIG. 6 is a drawing of an exploded view of the heat management system 530 shown in FIG. 5A.

In this embodiment, the heat management system 530 includes a heat sink 542, a heat spreader 543, one or more standoffs 544, a first thermally insulative layer 545 (e.g., a cork layer such as a 1 mm layer of cork), a second thermally insulative layer 546 (e.g., a neoprene or silicone layer), and a light detector 536. The light detector 536 includes a board camera 537 with a CMOS image sensor 538. In one implementation, the second thermally insulative layer 546 is a silicone layer that can allow for easy and thorough sanitization. The heat spreader 543 is located adjacent to the heat sink 542. The heat spreader 543 includes a plate of thermally conductive material (e.g., copper) that is U-shaped such that a USB-board camera 547 with the CMOS image sensor 538 may be located between the two outer walls 592, 594 to enable extraction of heat away from the USB-board camera 537 to the heat sink 542. The heat sink 542 is made of a thermally conductive material (e.g., an anodized aluminum) that includes a plurality of prongs or teeth for conducting heat to the ambient environment. The light detector package 430 also includes a housing 541 (e.g., a 3-D printed structure) within which components of the heat management system 530 and the light detector 536 may be contained and/or mounted.

FIG. 7 depicts a block diagram of a SCOS system 700 for determining cerebral blood metrics, according to various embodiments. System 700 includes a headset apparatus 720 with one or more channels (e.g., 1, 2, 3, 4, 5, 6, or more channels). The headset apparatus 720 may include a headband that can encircle a head of a user 701. User 701 (denoted as optional by aSCOS system 700 also includes a power source 780 (e.g., a 9 v bolt battery or the like) in electrical communication with the at least one light source. The system 700 also includes one or more computing devices 790 in communication with the headset apparatus 720 to receive speckle contrast image data. In one implementation, the one or more computing devices may be integrated into the headset apparatus 720.

The computing device(s) 720 includes one or more processors or other circuitry 792 and a non-transitory computer readable medium 794. The computing device 790 also includes a display 791 that can provide a graphical user interface (GUI) (e.g., GUI 895 in FIG. 8A) that may be used to input data to control functions of the headset apparatus 710. The GUI can also be used to control visualizations of the cerebral blood metrics.

FIG. 8A depicts a schematic illustration of an isometric view of an example of a portable multi-channel SCOS system 800 for generating cerebral blood metrics data, according to some embodiments. FIG. 8B depicts a schematic illustration of a top view of the multi-channel SCOS system 800 shown in FIG. 8A. The multi-channel system 800 includes a headset 801 with a headband 802 that can encircle a head 20 of a user. The headset 801 includes six channels 841, 842, 843, 844, 845, and 846. Additional or fewer channels may be included in other implementations.

Each channel includes a source-detector pair. The first channel 841 includes a first source-detector pair including a first light source 811 (e.g., a laser that can emit light in a near-infrared or infrared wavelength) and a first detector 831, the second channel 842 includes a second source-detector pair including the first light source 811 and a second detector 832, the third channel 843 includes a third source-detector pair including a second light source 812 and a third detector 833, the fourth channel 844 includes a fourth source-detector pair including the second light source 812 and a fourth detector 834, the fifth channel 845 includes a fifth source-detector pair including a third light source 813 and a fifth detector 835, and the sixth channel 846 includes a sixth source-detector pair including the third light source 813 and a sixth detector 836. The source-detector pairs of the six channels may be located along the headband 802 to probe different regions of the brain simultaneously or at different times. For example, the source-detector pairs may be located to measure cerebral blood metrics at right and left frontal lobe regions, right and left parietal lobe regions, right and left temporal lobe regions, or the like. In one implementation, the multi-channel SCOS system 800 may be used to generate cerebral blood metrics data that can be used to detect traumatic brain injury. In some cases, the multi-channel SCOS system 800 is configured to be able to adjust the locations of the light emission packages with the light sources and the light detector packages with light detectors along the headband 802.

The multi-channel SCOS system 800 also includes a first power source 881 (e.g., a 9 v battery or the like) electrically coupled to the first light source 811, a second power source 882 electrically coupled to the second light source 812, and a third power source 883 electrically coupled to the third light source 813. In addition, the multi-channel SCOS system 810 includes a computing device 890 and electrical connectors 838 between the light sources and the computing device 890 and between the light detectors and the computing device 890. In other implementations, wireless communication may be employed. The computing device 890 may be an integral part of the multi-channel SCOS system 800 and the electrical connectors 838 are omitted or the computing device 890 may be a separate component as shown in FIGS. 8A and 9A.

The computing device 890 includes a display 891 for providing a graphical user interface (GUI) 895 that may be used to control functions of the multi-channel SCOS system 810. The GUI 895 may also be used for visualizations of the cerebral blood metrics. For instance, in FIG. 9A, an example of a GUI 895 is shown having plots of the time traces of the cerebral blood flow measured at the six regions of the brain being probed by the source-detector pairs of the six channels 841, 842, 843, 844, 845, and 846. FIG. 9B illustrates examples of plots of time traces of cerebral blood flow and cerebral blood volume measured by the six channels 841, 842, 843, 844, 845, and 846 of the multi-channel SCOS system 800 in FIGS. 8A and 8B. In some cases, the computing device 790 may be used to perform near real-time processing of the image data to extract the cerebral blood metrics which may eliminate the need for computing resources for storing the raw images.

FIG. 10A depicts a schematic illustration of an isometric view of an example of a SCOS system 1000 for generating cerebral blood metrics data in a compact format, according to some embodiments. FIG. 10B depicts a schematic illustration of a top view of the SCOS system 1000 in FIG. 10A. FIG. 10C depicts a schematic illustration of a side view of the SCOS system 1000 in FIG. 10A. SCOS system 1000 includes a laser diode 1014 and a light detector 1036 mounted to a circuit board 1002 (e.g., printed circuit board (PCB)) forming a source-detector package with a low profile. The source-detector package may include a housing enclosing more components of the SCOS system 1000 or another structure that can be utilized to attach the SCOS apparatus 1000 to a headset.

In some cases, multiple source-detector packages may be attached to a headset to form a multi-channel SCOS system, where each source-detector package may be a channel. In a modular format implementation, an SCOS apparatus may include a headset that can be customized to remove or add any number of SCOS apparatus 1000 to the headset to adjust the number of channels and/or move the SCOS apparatus(es) 1000 to sensor locations for evaluating cerebral blood metrics at particular region(s) of interest in the brain.

In certain implementations, the circuit board 1001 in FIG. 10A may have a length in a range of 3 cm and 5 cm and a width in a range of 3 cm and 5 cm. In one example, the circuit board 1002 has a length of 5 cm and a width of 3 cm. In one example, the thickness of the circuit board 1002 is about 1 mm. In some cases, the circuit board 1002 may be made of a flexible material.

The laser diode 1014 may be configured to emit light in a near-infrared or infrared wavelength. In one example, the laser diode 1014 may emit light at about 830 nm. In another example, the laser diode 1014 may emit light at about 785 nm. In one instance, the laser diode 1014 may be a single-mode continuous wave 785 nm laser diode (e.g., Thorlabs laser L785H1) that can deliver up to 200 mW. The light detector 1036 includes dimensions of length, l, and width, w. In the illustrated example, the light detector 1036 is elongated having a length dimension longer than the width dimension. In some cases, light detector 1036 may have a length, l, in a range of 15 mm and 25 mm a width in a range of 5 mm and 10 mm.

In some implementations, the light detector 1036 may be segmented into a plurality of detector segments that can simultaneously acquire images. In one implementation, the light detector 1036 may be comprised of a plurality of sensors (e.g., CMOS sensors) arranged along the length, l, that can capture image frames simultaneously of different regions having different depths based on the corresponding S-D distances from the light diode 1014. By way of example, the light detector 1036 may be comprised of three sensors arranged along the lengthwise dimension including a first sensor closest to the laser diode 1014, a second sensor, and third sensor furthest from the laser diode 1014. The third sensor may acquire images at a greater depth than the images acquired by the first sensor and the second sensor. In another implementation, the light detector may include a sensor with a two-dimensional array of pixels divided into different sets (segments) of pixels that can simultaneously acquire images of different regions. An illustration of an implementation with a segmented light detector is shown in FIGS. 10A, 10B, and 10C.

The SCOS system 1000 also includes a light block 1050 made of a wall of opaque material located between the laser diode 1014 and the light detector 1036 to help prevent cross talk. Some examples suitable opaque materials include black plastic, PLA, and 3D printing black resins. In other implementations, one or both the laser diode 1014 and the light detector 1036 may be enclosed within an enclosure of opaque material or other light blocking structure may be used. The light block 1050 may have a length in a range of 2 cm and 5 cm a height in a range of 3 mm and 1 cm. The SCOS system 1000 also includes a wireless transmitter 1087 for sending cerebral blood metrics data such as time traces via a wireless communication connection such as BLUETOOTH or wifi to an external computing device 1090 (e.g. a cell phone).

The SCOS system 1000 also includes an onboard computing device 1080 including one or more processing units or processors (e.g., an application-specific integrated circuit (ASIC) or a programmable logic device such as a field-programmable gate array (FPGA)) coupled to the circuit board 1002. The processing units or processors may be configured with instructions for performing one or more operations of the SCOS system 1000. For example, the one or more processing units or processors of the onboard computing device 1080 may be configured to analyze data from the light detector 1036, generate cerebral blood metric data, and/or provide data representative of the cerebral blood metrics such as time traces to a second computing device 1090. The SCOS system 1000 also includes a power source 1085 in electrical communication with the laser diode 1014 and the one or more processing units or processors 1080 for providing power. The power source 1085 may be a rechargeable battery, for example.

In various embodiments, a SCOS system for determining cerebral blood metrics includes one or more processing units or processors. A processor can be implemented as a microcontroller or as one or more logic devices including one or more application-specific integrated circuits (ASICs) or programmable logic devices, such as field-programmable gate arrays (FPGAs) or complex programmable logic devices. If implemented in a programmable logic device, the processor can be programmed into the programmable logic device as an intellectual property block or permanently formed in the programmable logic device as an embedded processor core. In some other implementations, a processor can be or can include a central processing unit, such as a single core or a multi-core processor. The processor is coupled to memory and/or memory may be integrated in the processor, for example, as a system-on-chip package, or in an embedded memory within a programmable logic device itself.

### III. Examples of Methods for Determining Cerebral Blood Metrics

In some implementations, cerebral blood metrics may include one or more of cerebral blood flow, cerebral blood volume, and heart rate. In some embodiments, cerebral blood volume may be determined based on the intensity of reflected light as measured by a light detector. Note that cerebral blood volume may be determined based on reflected light of a single wavelength (e.g., emitted from a laser).

In some embodiments, cerebral blood flow may be determined using collected scattered light at a single wavelength (e.g., light emitted in an infrared or near infrared wavelength range). The wavelength implemented may affect penetration depth of the light. For example, light emitted in the infrared wavelength range may penetrate to deeper structures relative to near infrared light. In some implementations, cerebral blood flow measurements may be based on speckle as speckle contrast optical spectroscopy (SCOS) (sometimes referred to herein as visibility spectroscopy (SVS)). In general, a “speckle” refers to the pattern of bright and dark spots in an image resulting from scattering of illuminated laser light (e.g., scattered by the scalp, skull, and/or brain) resulting from constructive and destructive interference of the light. The speckle pattern dynamics change with blood flow dynamics. The time that it takes one speckle pattern to change to a different speckle pattern is referred to as the decorrelation time, which is correlated with cerebral blood flow rate. SCOS techniques measure how fast the speckle pattern changes, and hence, estimating a cerebral blood flow rate. SCOS typically determines speckle decorrelation time using a relatively slow camera with a large number of pixels. In some cases, the camera exposure time, typically in the range of 0.3 milliseconds to 15 milliseconds, may be set to be substantially longer than the decorrelation time of the speckle field (typically a few tens of microseconds), which may result in multiple different speckle patterns summed up into a single camera frame. As the speckle field fluctuates, the speckle pattern recorded by the camera is smeared and washed out within the exposure time. Accordingly, the cerebral blood flow may be quantified from the degree of blurring of the captured frame, which is generally referred to herein as the speckle contrast.

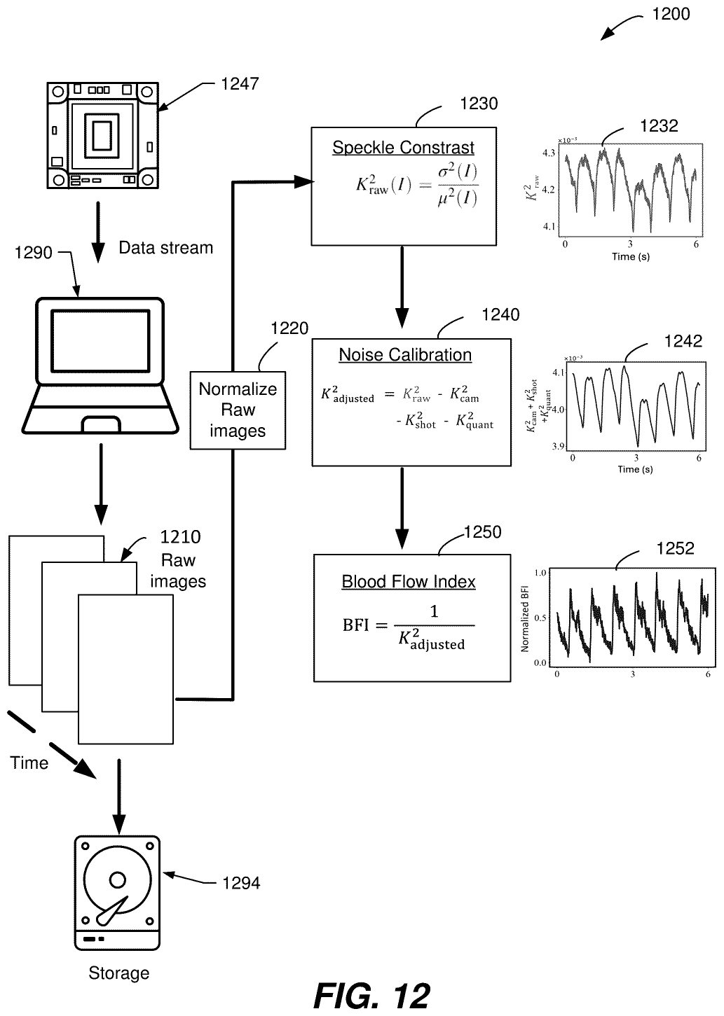

In one implementation, raw images are normalized to remove nonuniform intensity distributions. A speckle contrast is calculated from the normalized images and the calculated speckle contrast is adjusted to remove the influence of camera noise, shot noise, and quantization noise. The adjusted speckle contrast can be used to determine the cerebral blood flow measured in units of a normalized blood flow index. An example of a process flow for this implementation is shown in FIG. 12.

In one implementation, a raw speckle contrast is calculated directly from the raw speckle images. The raw speckle contrast is adjusted to remove the influence of camera noise, shot noise, quantization noise, and spatial inhomogeneity. The adjusted speckle contrast can be used to determine the cerebral blood flow measured in units of a blood flow index. An example of a process flow for this implementation is shown in FIG. 13.

Blood flow index (BFI) is typically used as a relative metric of blood flow velocity. Blood flow is generally related to blood pressure, the radius of the blood vessel, dynamic viscosity of the blood, and the length of the blood vessel. Any alteration in blood flow means a change in either the blood pressure or diameter of the blood vessel. A slight adjustment in the radius of the blood vessel can substantially impact blood flow due to the fourth power relationship of blood flow with the radius of the blood vessel. Comparing BFI values collected at different time points can serve as an indicator of changes in blood flow velocity.

BFI is a relative metric that is not generally used to determine blood flow directly for different conditions, including but not limited to, different S-D distances, different locations on the head or different subjects, and illumination wavelengths. This is because t can be influenced not only by the velocity of blood flow (BFI) but also by the blood flow volume (BFV), the presence of local vascular structures, and the blood pressure. Therefore, the determination of BFI from K²_{adjusted} is a relative approach and the determined BFI may be denoted as a relative blood flow index (rBFI) or as a normalized BFI.

The use of SCOS to measure cerebral blood flow may have the advantage that a relatively inexpensive camera may be used as a light detector, because a high frame rate is not needed. Moreover, a camera may be directly mounted on a headband, which may eliminate optical loss associated with a light guide running from the head to an external camera, which may introduce its own noise and motion artifacts. In some examples, a camera may have an integration exposure time of within a range of about 0.3 milliseconds-15 milliseconds. The camera may use a frame rate of within a range of about 30 frames per second-150 frames per second. In one example, the camera may have a frame rate of about 80 frames per second. In one example, the camera may have a frame rate of about 50 frames per second. Moreover, optical fibers are generally limited to 600 or 1000-microns in diameter whereas CMOS sensor sizes can be a square of dimension ranging from 1 mm to 10 mm, which is significantly larger. Positioning CMOS sensors directly atop the region of interest not only provides a stable placement but also provides a larger collection area and numerical aperture for collecting photons. Also, sensors may be divided into segments that allow a single light detector to capture multiple images at different S-D distances. For example, the light detector 1036 in FIGS. 10A-C may be divided into multiple segments (e.g., two segments, three segments, four segments, etc.).

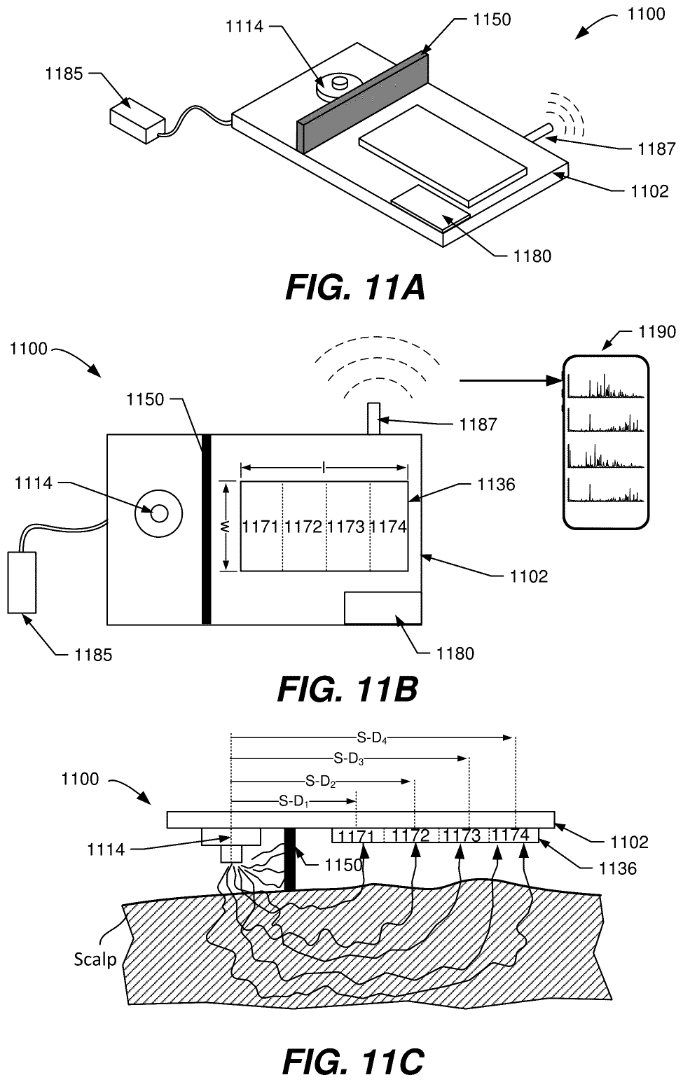

FIG. 11A depicts a schematic illustration of an isometric view of an example of a multi-channel SCOS system 1100 for generating cerebral blood metrics data in a compact format with a segmented light detector 1136, according to some embodiments. FIG. 11B depicts a schematic illustration of a top view of the multi-channel SCOS system 1100 in FIG. 11A. FIG. 11C depicts a schematic illustration of a side view of the multi-channel SCOS system 1100 in FIG. 11A. Some of the elements shown in FIGS. 11A, 11B, and 11C are similar or analogous to elements shown in FIGS. 10A, 10B, and 10C. For the sake of brevity, the prior discussion of such similar or analogous elements with regard to FIGS. 10A, 10B, and 10C may be assumed to be equally applicable, unless indicated otherwise, to the similar or analogous counterparts of those elements in FIGS. 11A, 11B, and 11C that share the same last two digits in their respective callouts as in FIGS. 10A, 10B, and 10C.

Multi-channel SCOS system 1100 includes a laser diode 1114 and a segmented light detector 1136 mounted to a circuit board 1102 forming a source-detector package with a low profile. The source-detector package may include a housing that can be utilized to attach the Multi-channel SCOS system 1100 to a headband of a headset. The segmented light detector 1136 includes a first segment 1171, a second segment 1172, a third segment 1173, and a fourth segment 1174. Each of these segments 1171, 1172, 1173, and 1174 can function as a separate channel. As depicted in FIG. 11C, the segments 1171, 1172, 1173, and 1174 can simultaneously acquire speckle images from different regions within the brain based on corresponding S-D distances S-D₁, S-D₂, S-D₃, and S-D₄ from the light diode 1114.

In addition to monitoring cerebral blood flow, SCOS systems described herein may also analyze intensity changes in the images captured by the light detector to take absorption measurements that can be used to determine changes in cerebral blood volume (CBV). In some cases, the light detector is configured to emit an illumination wavelength of 785 nm, which may minimize the influence of oxygen concentration changes.

### Example 1

FIG. 12 depicts an example of a process flow for determining cerebral blood flow and/or cerebral blood volume from raw speckle images 1210, according to embodiments. Components of a SCOS system for determining cerebral blood metrics (e.g., SCOS system 100 in FIG. 1, multi-channel SCOS system 810 in FIGS. 8A and 8B, SCOS system 900 in FIG. 9, etc.) may be used to implement the process flow. The cerebral blood flow may be measured in units of blood flow index (BFI). The cerebral blood volume may be measured in units of blood flow index (BVI). As shown, a light detector 1247 (e.g., a camera) of the SCOS system captures a plurality of raw images 1210. The exposure time of the light detector 1247 is substantially longer than the decorrelation time of the speckle field such that multiple speckle patterns are integrated into each raw speckle image. The raw images 1210 may be stored to and retrieved from a non-transitory computer readable medium 1294.

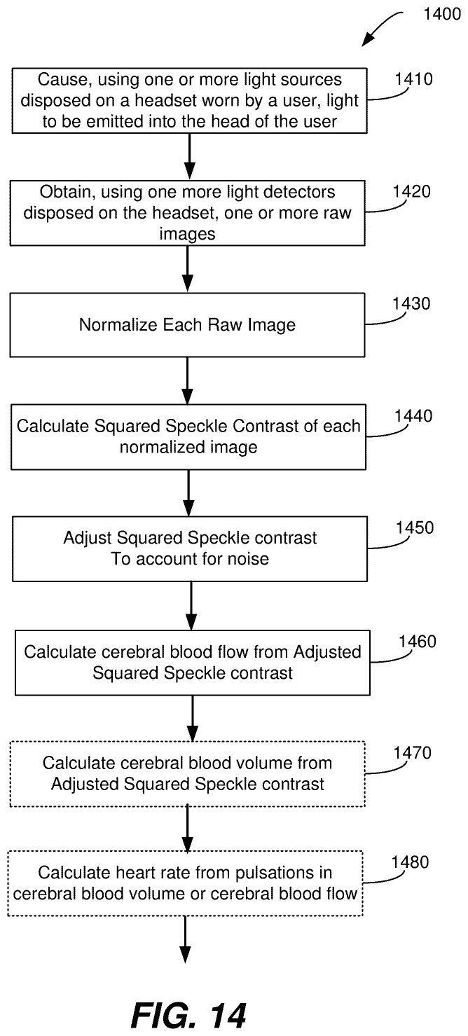

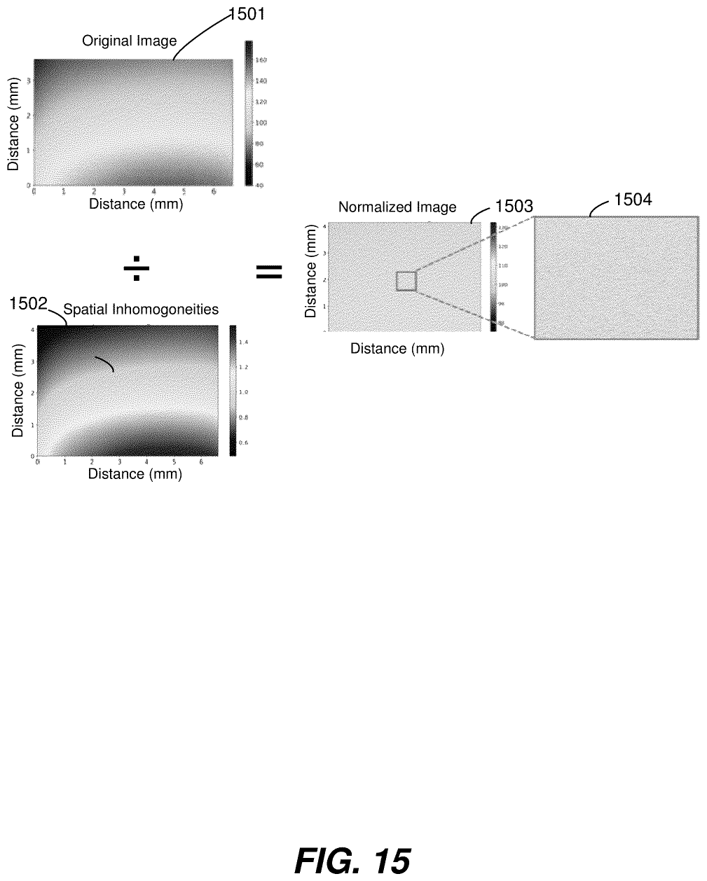

The raw images may have nonuniform intensity distributions. For example, since one side of the light detector is closer to the light source, the pixels closer to the light source will have higher intensities than those further away. To remove the nonuniform intensity distribution from the images, each raw image is normalized (block 1220) by dividing each raw image by a mean image. The mean image is determined from a plurality of raw images acquired in a calibration operation, typically taking place in a time period before the plurality of raw images 1210 are acquired. An example of a normalization operation is described in more detail with reference to FIG. 14.

At block 1230, the squared speckle contrast may be determined at each normalized image, I, as:

$${K}_{raw}^{2} ( I ) = \frac{{σ}^{2} ( I )}{{μ}^{2} ( I )} \qquad ( Eqn . 1 )$$

In Eqn. 1, σ² (I) represents the variance of the normalized image I at time t, and μ represents the mean of the normalized image I. An example of the squared speckle contrast as a function of time is shown in graph 1232.

At block 1240, the adjusted squared speckle contrast of each normalized image, I, that accounts for noise may be determined by:

$${K}_{adjusted}^{2} ( I ) = {K}_{raw}^{2} ( I ) - {K}_{shot}^{2} ( I ) - {K}_{q uant}^{2} ( I ) - {K}_{cam}^{2} ( I ) \qquad ( Eqn . 2 )$$

In Eqn. 2, K²ₛₕₒₜ (I) is the contribution to variance of the images from shot noise, K²_{quant} (I) is the contribution to variance of the images from quantization, and K²_{cam} (I) is the contribution to variance of the images from camera readout noise. An example of the contributions to variance from shot noise, camera noise, and quantization noise as a function of time is shown in graph 1242.

The contribution to variance of the images from shot noise, K²ₛₕₒₜ (I) may be determined for each normalized image as:

$${K}_{shot}^{2} ( I ) = \frac{γ}{μ ( I )} \qquad ( Eqn . 3 )$$

In Eqn. 3, γ is the analog to digital conversion ratio associated with the light detector 1247, which depends on the gain setting and the conversion factor (CF), and can be determined by:

$$γ = \frac{gain}{CF} \qquad ( Eqn . 4 )$$

The contribution to variance of the images from quantization, K²_{quant} (I) may be determined for each normalized image as:

$${K}_{quant}^{2} ( I ) = \frac{1}{12 {μ ( I )}^{2}} \qquad ( Eqn . 5 )$$

The contribution to variance of the images from camera noise, K²_{cam} (I) may be determined for each normalized image as:

$${K}_{cam}^{2} ( I ) = \frac{{σ}_{cam}^{2}}{{μ ( I )}^{2}} \qquad ( Eqn . 6 )$$

In Eqn. 6, σ²_{cam} is the camera noise, which can be estimated by calculating the variance of a plurality of raw images (e.g., 100, 200, 300, 400, 500, 600, etc.) acquired in the absence of light being emitted by the light source.

At block 1250, the cerebral blood flow index at the time of image acquisition, t, can be determined by:

$$BFI ( t ) = \frac{1}{{K}_{adjusted}^{2} ( I )} \qquad ( Eqn . 7 )$$

An example of the cerebral blood flow index as a function of time is shown in graph 1252.

Blood volume measurements are based on the light absorbed by the tissues being probed and can be determined from intensities in image frames. The cerebral blood volume may be measured in units of cerebral blood volume index (BVI). The cerebral blood volume index can be determined at each raw image acquisition time, t, as:

$$BVI ( t ) = \frac{2 {I}_{0} - 〈 I ( t ) 〉}{{I}_{0}} \qquad ( Eqn . 8 )$$

In Eqn. 8, I(t) is the mean pixel intensity value of the raw image and I₀ is the mean pixel intensity value of a baseline image taken during a baseline time period before the breath-holding time period.

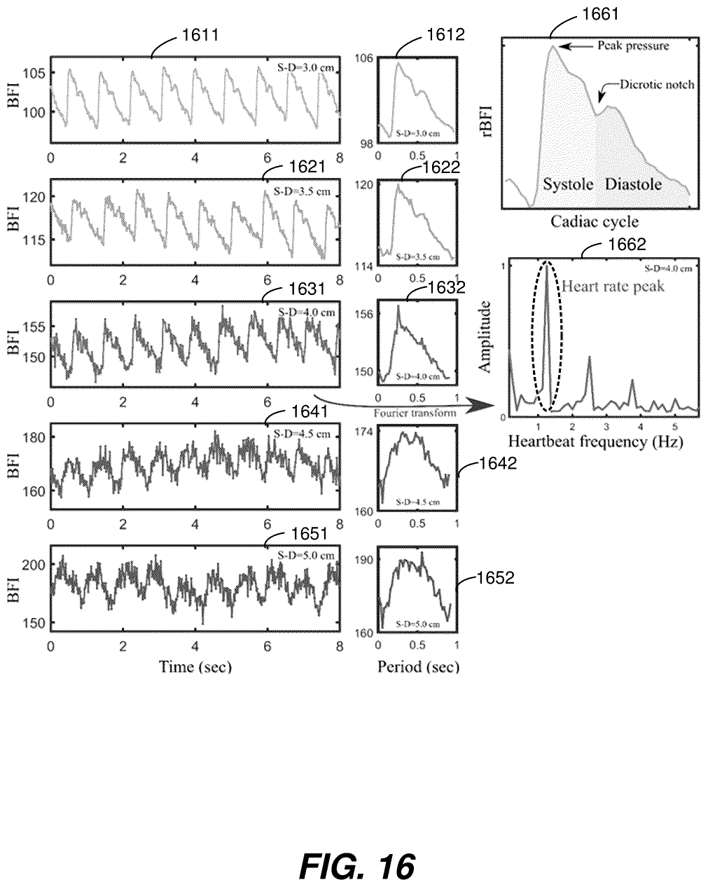

The heart rate can be determined from the pulsations in the blood flow or blood volume measurements over time, which is described in more detail below with reference to FIG. 13 and FIG. 16. For example, the heart rate may be determined by taking a Fourier transform of time domain data such as time traces of cerebral blood flow or cerebral blood volume. The heart rate can be determined as the first sub-harmonic peak of the pulsations in the frequency domain.

### Example 2

FIG. 13 depicts another example of a process flow for determining cerebral blood flow and/or cerebral blood volume, according to embodiments. Components of an apparatus for determining cerebral blood metrics (e.g., SCOS system 100 in FIG. 1, multi-channel SCOS system 810 in FIGS. 8A and 8B, etc.) may be used to implement the process flow. As shown, a light detector 1347 (e.g., camera) of the SCOS system captures a plurality of raw speckle images 1310. The exposure time of the light detector 1347 is substantially longer than the decorrelation time of the speckle field such that multiple speckle patterns are integrated into each raw speckle image. The images 1310 may be stored to and retrieved from a non-transitory computer readable medium 1394.

At block 1330, to quantify the fluctuations of the speckle field, the raw squared speckle contrast may be determined at each raw image as:

$${K}_{raw}^{2} = \frac{{σ}_{raw}^{2}}{{( {μ}_{raw} - {μ}_{offset} )}^{2}} \qquad ( Eqn . 9 )$$

In Eqn. 9, σ_{raw} is the standard deviation and μ_{raw} is the mean of the recorded image, and μ_{offset} accounts for the light detector offset. To experimentally measure the offset, a series of images are captured without any source illumination, and the mean value μ_{offset} is calculated of all the captured images. An example of the raw squared speckle contrast as a function of time is shown in graph 1332.

At block 1340, the adjusted raw squared speckle contrast of each raw speckle image that accounts for noise may be determined by:

$${K}_{adjusted}^{2} = {K}_{raw}^{2} - {K}_{shot}^{2} - {K}_{quant}^{2} - {K}_{cam}^{2} - {K}_{sp}^{2} \qquad ( Eqn . 10 )$$

In Eqn. 10, K²ₛₕₒₜ is the contribution to variance of the images from shot noise, K²_{quant} is the contribution to variance of the images from quantization, K²_{cam} is the contribution to variance of the images from camera readout noise, and K²ₛₚ is the contribution to variance of the images from spatial inhomogeneities. An example of the contributions to variance from this noise as a function of time is shown in graph 1342.

The contribution to variance of the images from shot noise, K²ₛₕₒₜ may be determined for each raw image as:

$${K}_{shot}^{2} = ( \frac{γ}{{μ}_{raw} - {μ}_{offset}} ) \qquad ( Eqn . 11 )$$

In Eqn. 11, the γ is the analog to digital conversion ratio associated with the light detector, which depends on the gain setting and CF of the light detector and can be determined by Eqn. 4. In some examples, the gain is set in a range of 1 to 72, corresponding to a 0 to 37 dB setting. In one example, the light detector 1347 is a Basler board camera with a conversion factor of CF=40.7.

The contribution to variance of the images from quantization, K²_{quant} may be determined for each raw image as:

$${K}_{quant}^{2} = ( \frac{1}{12 {( {μ}_{raw} - {μ}_{offset} )}^{2}} ) \qquad ( Eqn . 12 )$$

The contribution to variance of the images from camera noise, K²_{cam} may be determined for each raw image as:

$${K}_{cam}^{2} = ( \frac{{σ}_{cam}^{2}}{{( {μ}_{raw} - {μ}_{offset} )}^{2}} ) \qquad ( Eqn . 13 )$$

In Eqn. 13, σ²_{cam} is the camera noise, which can be estimated by calculating the variance of a plurality of raw images (e.g., 100, 200, 300, 400, 500, 600, etc.) acquired in the absence of light being emitted by the light source.

The contribution to variance of the images from spatial inhomogeneities, K²ₛₚ may be determined for each raw image as:

$${K}_{sp}^{2} = ( \frac{{σ}_{sp}^{2}}{{( {μ}_{raw} - {μ}_{offset} )}^{2}} ) \qquad ( Eqn . 14 )$$

In Eqn. 14, σ²ₛₚ is the noise from spatial variations in photon flux across the light detector area.

At block 1050, the relative cerebral flow can be determined as:

$$rCBF ∝ \frac{1}{τ} = \frac{1}{β {K}_{adjusted}^{2}} \qquad ( Eqn . 15 )$$

The speckle decorrelation time is directly correlated with the cerebral blood flow rate. The relative cerebral blood flow, measured in units of blood flow index (BFI), is inversely correlated with τ and therefore inversely correlated with βK². The correction factor β is a constant depending on system setup, e.g. speckle size, pixel size, polarization of the laser light.

The relative cerebral blood volume can be determined for each raw image as:

$$rCBV = \frac{1}{{μ}_{raw} - {μ}_{offset}} \qquad ( Eqn . 16 )$$

### Example 3

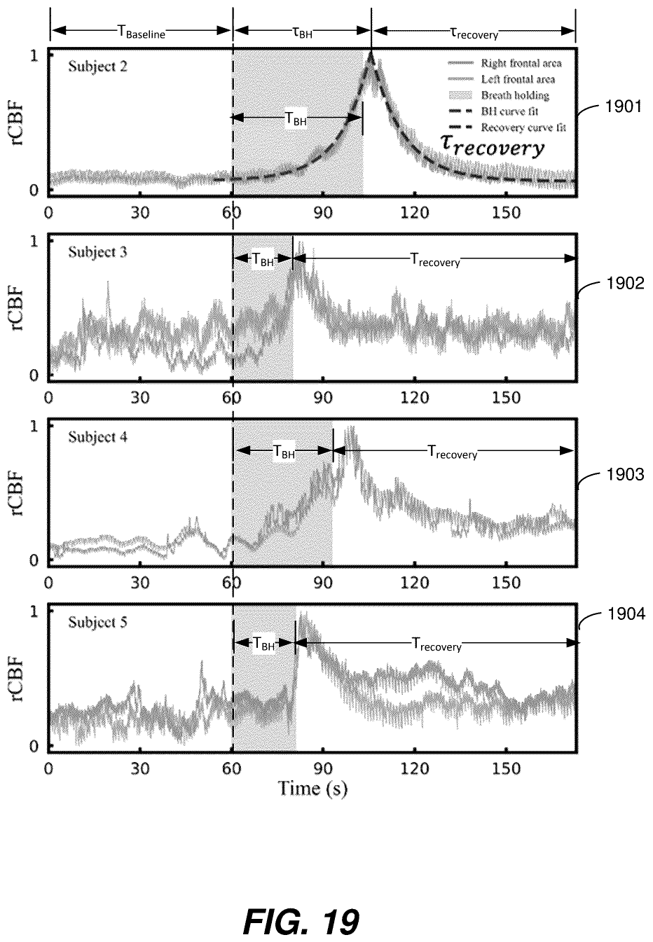

FIG. 14 is a flowchart of an example process for determining cerebral blood metrics in accordance with some embodiments. Blocks of process 1400 may be executed by one or more processors of one or more computing devices. An example of such a computing device is shown in and described below in connection with FIG. 19. Note that, in some embodiments, at least one of the one or more computing devices may be disposed on the headset on which the one or more light sources and light detectors are disposed. For example, the SCOS system 800 with computing device 880 may be attached to a headset. Accordingly, in some such embodiments, cerebral blood metrics may be determined by a computing device itself on the headband or headset. Alternatively, in some embodiments, data obtained by the one or more light detectors, and/or data representative of the cerebral blood metrics may be transmitted from a computing device disposed on the headband or headset to a second computing device remote from or separate from the headband or headset, where the second computing device generates cerebral blood metrics. In some implementations, blocks of process 1400 may be executed in an order other than what is shown in FIG. 14. In some embodiments, one or more blocks of process 1400 may be omitted, and/or two or more blocks may be executed substantially in parallel.

At 1410, process 1400 can begin by causing, using one or more light sources disposed on a headset worn by a user, light to be emitted into the head of the user. Examples of such a headset are shown in and described above in connection with FIGS. 1, 2, and 7. The one or more light sources may include one or more lasers, each of which may emit light in the near infrared or infrared wavelength. In some implementations, multiple light sources are disposed on a headset or headband, each configured to emit light into different regions of the user's head or brain. The light may be emitted continuously, or may be pulsed.

At 1410, process 1400 may obtain, using one or more light detectors disposed on the headset, information indicative of light reflected from one or more structures within the head or brain of the user. The obtained information includes image data for a plurality of raw images. Each raw image is captured over an exposure time that may be substantially longer than the decorrelation time of the speckle field such that multiple speckle patterns are integrated into each raw image. An example of one or more light detectors disposed on a headset is shown in and described above in connection with FIGS. 1, 2, and 8. As described above, the one or more light detectors may include one or more CMOS sensors, a camera, etc. In some cases, a portion of the obtained information spans a time period during which the user was holding their breath.

In one example, light emitted by a laser may be reflected from various head and brain structures and may be captured by a camera (e.g., to determine speckle contrast, as described above). Note that, as shown in and described above in connection with FIG. 8A, because a headset may include multiple (e.g., two, four, eight, ten, etc.) light emission packages and light detector packages, the obtained information may correspond to different brain regions (e.g., a left frontal lobe region, a right frontal lobe region, a left parietal lobe region, a right parietal lobe region, etc.).

Note that, the obtained light reflection data may span a time period that includes a baseline time period, a breath holding time period, and a recovery time period. In some implementations, process 1400 may cue the user to begin holding their breath at a particular time. For example, the cue may be an audible cue (e.g., a spoken instruction, an audible beep, etc.), or may be a haptic cue (e.g., a vibration delivered using the headset).

At 1420, process 1400 may, normalize each raw image by dividing each raw image by a mean image. The mean image refers to an averaged intensity distribution of a plurality of images obtained during a time period before the time period used to acquire the plurality of speckle images. For example, the plurality of raw images may be obtained in a time period during which the user was holding their breath (breath hold period). The mean image is determined from another plurality of images (e.g., 50, 100, 200, 300, 400, 500, etc.) obtained during a baseline period prior to the breath hold period during.

At 1430, process 1400 calculates the speckle contrast of each normalized image using Eqn. 1. At 1440, process 1400 adjust the speckle contrast to account for noise and other factors. For example, the speckle contrast may be adjusted to remove variance due to shot noise, camera noise, and quantization as provided in Eqn. 2. Contribution to variance of the images from shot noise may be determined for each normalized image by Eqn. 3. Contribution to variance of the images from quantization may be determined for each normalized image by Eqn. 5. Contribution to variance of the images from camera noise may be determined for each normalized image by Eqn. 6.

At 1450, process 1400 determines the cerebral blood flow from the adjusted speckle contrast. For example, the process 1400 may determine the cerebral blood flow in units of blood flow index using Eqn. 7.

Blood volume measurements are based on the light absorbed by the blood and tissues being probed and can be determined from intensities in image frames. Assuming the tissues will not change, the only changes in absorption are due to increasing or decreasing blood, which can be inferred as relative changes in blood volume. At optional (denoted by dashed line) 1460, process 1400 may determine the cerebral blood volume at each image acquisition time using Eqn. 8.

At optional (denoted by dashed line) 1470, process 1400 may determine the heart rate based on the periodicity of pulsations in time domain data of cerebral blood volume or the cerebral blood flow measurements. For example, the plots 1611, 1621, 1631, 1641, and 1651 in FIG. 16 are time traces of cerebral blood flow measurements that have blood pulsations. The process 1400 can take a Fourier transform of the traces and determine the first harmonic peak in the frequency domain as the heart rate.

### V. Experimental Data

### A. S-D Distances

The distances between a light source and a light detector (S-D distances) impact the depth emitted light can penetrate within the brain and the corresponding depths of imaging and signal sensitivity. The scalp and skin of the user as well as other conditions may also affect signal sensitivity. In certain implementations, the S-D distance for a particular subject may be tuned for signal sensitivity. In some cases, an S-D distance of between 3 cm and 4.5 cm may provide an adequate signal sensitivity to detect cerebral blood flow.

The SCOS system 100 in FIGS. 1A and 1B was used to obtain cerebral blood metrics data from ten subjects. The headset 101 was placed on the forehead of the subjects as shown in FIG. 1. Measurements were collected over 8 seconds for each of five different source-to-detector (S-D) distances (±2 mm): 3.0 cm, 3.5 cm, 4.0 cm, 4.5 cm, and 5.0 cm for each subject.

FIG. 16 illustrates examples of experimental cerebral blood metric data collected over 8 seconds for five different source-to-detector (S-D) distances (±2 mm): 3.0 cm, 3.5 cm, 4.0 cm, 4.5 cm, and 5.0 cm for a first subject. As illustrated by graphs 1611, 1621, 1631, 1641, and 1651, cerebral blood flow is monitored in units of blood flow index during an 8 second time period by a light detector at source-to-detector (S-D) distances of 3.0 cm, 3.5 cm, 4.0 cm, 4.5 cm, and 5.0 cm. In this example data for the first subject, an S-D distance of 3 cm provided adequate brain signal sensitivity to detect cerebral blood flow. As the S-D distance extended beyond 3.0 cm, the gain in sensitivity becomes progressively smaller, peaking at S-D distances of approximately 4 cm.

Plots 1612, 1622, 1632, 1642, and 1652 illustrate the average cardiac cycle of the blood flow index measurements in graphs 1611, 1621, 1631, 1641, and 1651. To determine the average cardiac cycle in plot 1612, the BFI signal in 1611 was partitioned into windows of duration of 1/HR seconds (HR being the heartrate frequency from graph 1662) and subsequently calculating the mean value across all these windows. Graph 1661 illustrates two peaks, labeled peak pressure and dicrotic notch, within the plot 1612 of the average cardiac cycle of the blood flow index measurements in graphs 1611. In general, peak pressure corresponds to the rapid ejection of blood during systole and the dicrotic notch is at the beginning of diastole.

The pulsations evident in each of graphs 1611, 1621, 1631, 1641, and 1651 represent blood pulsations. These pulsations may be used to determine a heart rate of the subject (e.g., based on the periodicity of the pulsations). For example, the heart rate may be determined by taking a Fourier transform of time domain data. For instance, the frequency domain graph 1662 of heartbeat frequency is a Fourier transform of the time domain data in graph 1631 of cerebral blood flow index measurements. The heart rate may be determined based on the heartbeat peak frequency (first sub-harmonic) 1663 in the frequency domain graph 1662. In the illustrated example, the heartbeat of the subject was measured at heart rate (HR)=1.25 Hz (75 bpm).

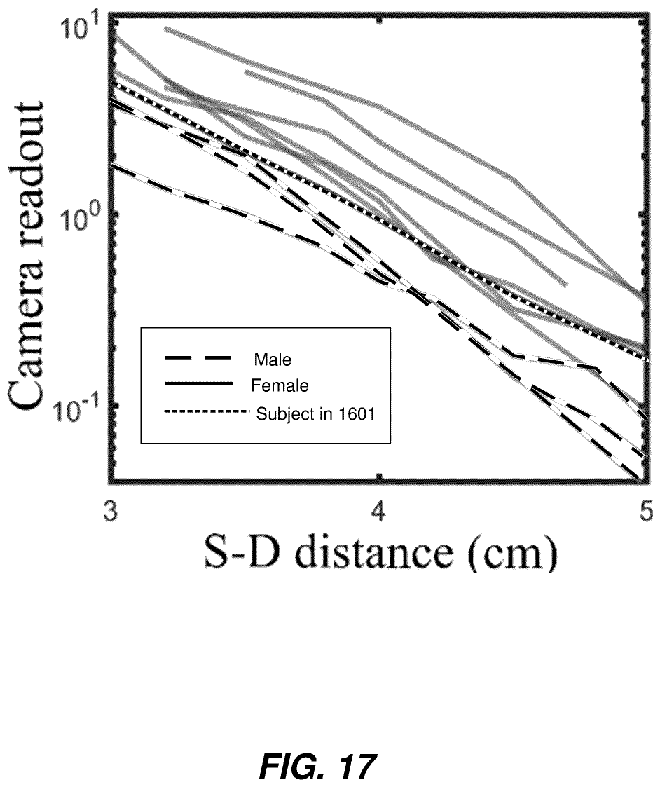

The headset 101 from FIGS. 1A and 1B was then used to obtain cerebral blood metric data for an additional nine subjects. The measurements were taken during an 8 second time period by a light detector at source-to-detector (S-D) distances of 3.0 cm, 3.5 cm, 4.0 cm, 4.5 cm, and 5.0 cm. FIG. 17 depicts a plot 1701 of the average measured camera intensity as a function of the S-D distances for the ten subjects. An average measured camera intensity at each S-D distance can be calculated by taking the average intensity of all the raw images taken at that distance. The data shows that for S-D distances up to 4.5 cm for all subjects, the readout intensities were adequate (above noise level) to obtain blood metric data.

### B. Breath Holding for Cerebrovascular Assessment

The brain is highly complex and plays a significant role in overseeing vital bodily functions such as the rate of oxygenation and blood circulation. Those are controlled by the brain by modulating the depth and pace of breathing and by regulating blood pressure by fine-tuning both heart rate and blood vessel diameter. The brain regulates its own blood supply, ensuring a consistent delivery of oxygen and nutrients. Together, these mechanisms guarantee the maintenance of physiological conditions.

The SCOS system for determining cerebral blood metrics of certain implementations can be used to assess cerebrovascular reactivity, for example, by measuring the ability of the brain to adjust cerebral blood flow in response to changing oxygen supply in the body. In some examples, the SCOS system can monitor temporal cerebral blood flow changes during controlled breath modulation tasks to be able to evaluate dynamic cerebrovascular responses elicited by oxygen deprivation scenarios.

During a breath holding time period, the brain enters a heightened state of alert which triggers a sequence of protective mechanisms to ensure stable regulation of carbon dioxide and oxygen, which is achieved through accelerated circulation of blood leading to increased blood flow together with an increase in blood volume in the brain via dilation of blood vessels. At the beginning of a breath hold, the cerebral blood flow swiftly increases although with some fluctuation. As the breath hold prolongs, cerebrovascular reactivity should exhibit escalated demand for increased blood flow to facilitate oxygen transport. When the subject concludes the breath holding phase and resumes normal respiration, the surplus oxygen in the brain is released, and cerebral activity returns to its previous stable state.

Cerebral blood metrics may be obtained over a breath holding time period during which the patient is holding their breath. Examples of breath holding time periods, T_{BH}, are illustrated in FIG. 19. Cerebral blood metrics may also be obtained over a baseline time period, generally referring to the time period during which cerebral blood metrics are obtained prior to initiation of breath holding. Examples of baseline time periods, T_{Baseline}, are illustrated in FIG. 19. Cerebral blood metrics may also be obtained during a recovery time period, which generally begins at a time from when breath holding ends (e.g., the end of breath holding time period). Examples of recore4y time periods, T_{recovery}, are illustrated in FIG. 19. Note that each time period may be any suitable duration of time. Example duration of breath holding may be 15 seconds, 30 seconds, 45 seconds, 60 seconds, etc. In some embodiments, the duration of breath holding may be however as long a patient can hold their breath relatively comfortably (e.g., the breath holding time period may vary for different people). In some implementations, the baseline time period and/or the recovery time period may be at least as long as the breath holding time period. In some embodiments, the recovery time period may be longer than the breath holding time period. In some embodiments, the recovery time period may be a time duration long enough that cerebral blood metrics return to within a predetermined range of the corresponding values during the baseline time period. In some such embodiments, the recovery time period may be dynamically adjusted. For example, the recovery time period may be stopped responsive to determined that cerebral blood metrics have returned to baseline values.

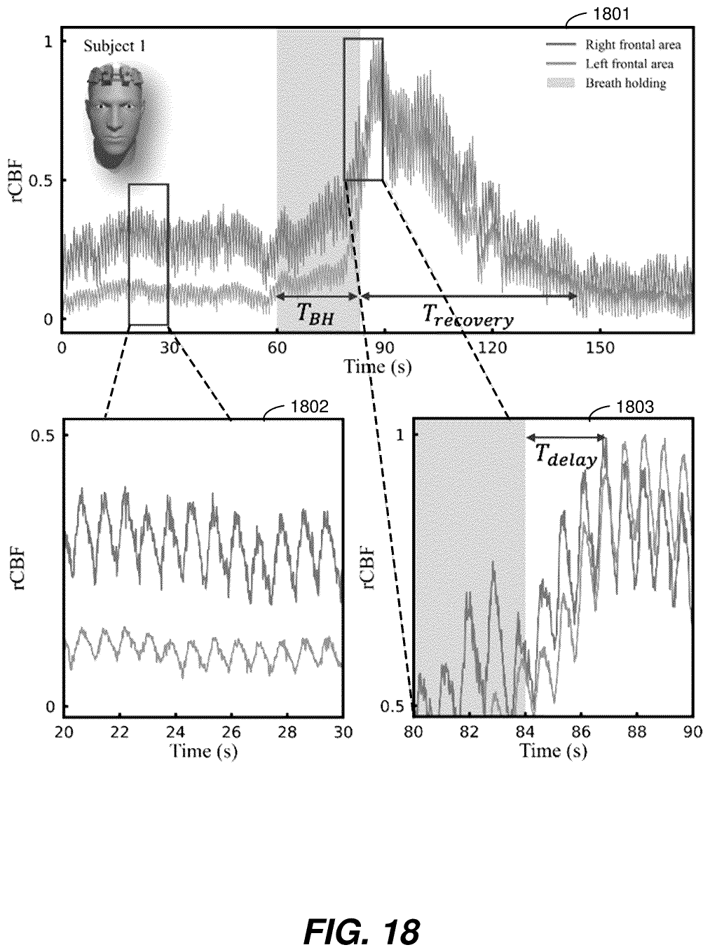

As depicted in an inset in the illustration 1801 in FIG. 18, a multi-channel SCOS system 1820 with two channels was used to collect cerebral blood flow data during a breath holding task in accordance with some embodiments. Measurements of cerebral blood flow were taken at two distinct locations on the subject's forehead as illustrated.

FIG. 18 illustrates examples of experimental relative cerebral blood flow data collected during a breath holding task in accordance with some embodiments. As illustrated by a relative cerebral blood flow graph 1801, cerebral blood flow is determined during a time period that includes baseline time period 1810 during which the subject is breathing normally, a breath holding time period 1811 during which time the patient is holding their breath, and a recovery time period 1812 after the patient resumes normal breathing, and a post-recovery time period 1813 after the recovery time period. For the breath holding task, a subject is asked to breathe normally for 60 seconds. At the 60th second, the subject was prompted to fully exhale and then voluntarily hold their breath for as long as deemed comfortable. Graph 1802 illustrates a subset of the relative cerebral blood flow graph 1801 over a short time scale during the baseline time period. Graph 1803 illustrates a subset of the relative cerebral blood flow graph 1801 over a short time scale during the breath hold time period. The high frequency fluctuations in the time trace of the relative cerebral blood flow data are cardiac blood pulsations, not noise.

As illustrated in graphs 1801 and 1803, the relative cerebral blood flow increases during breath holding time period 1811. This is attributed to the brain's increased demand for blood to transport oxygen and carbon dioxide until breath holding stops. During the breath holding time period 1811, the brain enters a heightened state of alert which triggers a sequence of protective mechanisms to ensure stable regulation of carbon dioxide and oxygen, which is achieved through accelerated circulation of blood leading to increased blood flow together with an increase in blood volume in the brain via dilation of blood vessels.

As the duration of breath hold prolongs, the brain exhibits an escalated demand for increased blood flow to facilitate oxygen transport, until breath hold task stops. Additionally, note that two to ten seconds after termination of the breath hold, the cerebrovascular reactivity still exhibits a heightened reaction, and the relative cerebral blood flow continues to increase at the beginning of the recovery time period 1812. After normal breathing resumes, the cerebral blood flow level returns to its previous baseline state.

FIG. 19 illustrates examples of experimental relative cerebral blood flow data collected during a breath holding task for four subjects, in accordance with some embodiments. As illustrated by relative cerebral blood flow graphs 1901, 1902, 1903, and 1904, cerebral blood flow is determined during a time period that includes a baseline time period (T_{Baseline}) during which the subject is breathing normally, a breath holding time period (T_{BH}) during which time the patient is holding their breath, and a recovery time period after the patient resumes normal breathing.

Various cerebral blood metrics data obtained during a breath hold task may be used as indicators of cerebrovascular health. One example metric is a duration of time a subject was able to hold their breath (generally referred to herein as T_{BH}). The range of T_{BH} can vary significantly between subjects due to differences in physical conditions and tolerance levels. An example of a T_{BH} is shown in graph 1801. Another example metric is a duration of time required for the cerebral blood flow to return to pre breath holding baseline, referred to as T_{recovery}. An example of a T_{recovery} is shown in graph 1801. Two other example metrices are T_{recovery} and τ_{BH}, which are measured by fitting the breath holding cerebral blood flow data with an exponential curve

$$( C B F = a \cdot exp ( \pm \frac{t}{τ} ) + c )$$

as illustrated in the graph 1901 shown in FIG. 19. Another example metric is the duration of time between the end of the breath holding period and the peak of the cerebral blood flow trace, referred to as T_{delay}. An example of a T_{delay} is shown in graph 1803. T_{delay} may serve as an indicator of the brain's response rate to abrupt changes in the body's oxygen supply. Other metrics may include a percentage change in a cerebral blood metric at a maximum or minimum after initiation of breath holding compared to a baseline value. For example, the percentage change in cerebral blood volume ((BV change) and the percentage change in cerebral blood flow (CBF change). Note that to determine a percentage change, each cerebral blood metric may be normalized based on the baseline value. Other metrics include a rate of change (e.g., a slope) in the change in a cerebral blood metric during breath holding. For example, a rate of change of cerebral blood flow may be determined by dividing a percentage change of cerebral blood flow by the duration of time the subject holds their breath, to derive a feature with units of percent change per second. Similarly, a metric may include a rate of recovery (e.g., a slope) in the change in a cerebral blood metric after resuming normal breathing. Note that, in some implementations, rates of change either during the breath holding time period, or a rate of change associated with recovery, may be determined by fitting a function (e.g., an exponential function) to a portion of the cerebral blood metric, and estimating rate of change metrics based on a growth or decay constants from the fitted function.

In some implementations, metrics may include a ratio of a percentage change of one cerebral blood metric to a percentage change of another cerebral blood metric. In one example, a metric may be a ratio of the percentage change of cerebral blood flow to the percentage change of cerebral blood volume. In some implementations, the metric may include a ratio of rate of change of one cerebral blood metric to a rate of change of another cerebral blood metric. An example includes a rate of change of cerebral blood flow to a rate of change of cerebral blood volume. Note that ratios of rates of change may be determined based on rate of change either during the breath holding time period, or during a recovery time period after normal breathing resumes.

### C. Cerebral Blood Volume and Heart Rate

In addition to measuring cerebral blood flow, the various SCOS systems described can also measure cerebral blood volume and heart rate. The heart rate can be determined based on pulsations in traces of cerebral blood flow or cerebral blood volume as discussed above with reference to FIG. 16. Cerebral blood volume measurements are based on the light absorbed by the brain and can be determined from intensities in image frames. The cerebral blood volume may be determined using Eqn. 8 or Eqn. 15.

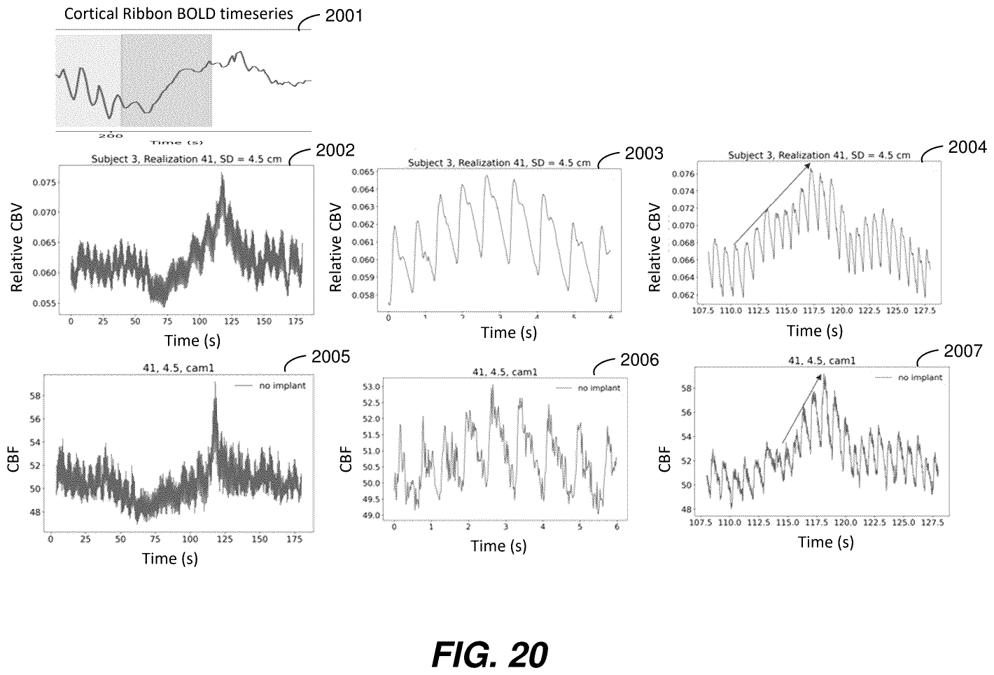

FIG. 20 illustrates examples of graphs of cerebral blood flow and cerebral blood volume in accordance with some embodiments and MRI data for comparison. In MRI experiments, subjects are asked to hold their breath for 20 seconds, followed by a 1-minute recovery window. The MRI acquired BOLD signal result is shown in graph 2001. In correspondence, the SCOS device was placed on a subject with one channel on the left portion of the forehead. In the SCOS experiment, the subject was asked to hold breath voluntarily starting from 60-second mark, with the total acquisition duration set at 3 minutes. In this case, the subject held their breath from 60 to 115 seconds. The breath-holding task was repeated five times, with results from one of the instances displayed in graphs 2002-2007 shown in FIG. 20. Both CBV (graphs 2002-2004) and CBF (graphs 2005-2007) show a similar trend in comparison to the MRI acquired BOLD signal. When zoomed in, the blood flow dynamics within a cardiac period can be observed, e.g. graph 2006. CBF traces will demonstrate additional higher frequency details in comparison to CBV. Based on overall trend, the CBF traces also have sharper increase towards the end of the breath holding, e.g. as shown in graphs 2005 and 2007, in comparison to the CBV, e.g. as shown in graphs 2002 and 2004. This points to stressing to the limit of cerebral autoregulation where blood vessels cannot further expand to accommodate the blood pressure, thus the sudden increase in CBF towards the end of the breath holding.

### D. Experimental Data in Clinical Setting

An implementation of the portable multi-channel SCOS system 800 with six channels 841, 842, 843, 844, 845, and 846 shown in FIGS. 8A and 8B was used to simultaneously measure CBF and CBV at six corresponding brain locations of subjects in a clinical setting. The measurements were taken while the subjects were located in hospital rooms to demonstrate use of the portable multi-channel SCOS system 800 in clinical patients in care. More specifically, the portable multi-channel SCOS system 800 was used to simultaneously measure CBF and CBV at six different brain locations of (I) five healthy subjects, and (II) two subjects with brain injury, one with traumatic brain injury (TBI) and one with non-traumatic brain injury (NTBI). The NTBI subject showed evidence of structural brain damage. This implementation of the portable multi-channel SCOS system 800 included three light sources 811, 812, and 813 in the form of laser diodes and six light detectors 831, 832, 833, 834, 835, and 836 in the form of USB board cameras, which were integrated into a wearable headset 801 along with power sources 881, 882, and 883 in the form of rechargeable 9V batteries. The power sources 881, 882, and 883 were embedded in the headset 801. Data was transmitted via electrical connectors 838 in the form of a USB cable to the computing device 890 for near real-time processing, which may enable operation in diverse environments. For near real-time processing, upon completion of recording, the GUI immediately presents CBF and CBV for each channel. The near real-time processing of data may negate the need to save the raw speckle images, thereby reducing disk space usage. Each of the light sources 811, 812, and 813 was a single-mode continuous wave laser diode of 808 nm (e.g., Thorlabs M9 808-0150). To control the illumination spot size and avoid laser light reflections or stray light, the laser diode was housed (e.g., housing 311 in FIGS. 3A and 3B) in a 3D-printed mount with a circular aperture of 5.5 mm diameter. The portable multi-channel SCOS system 800 included a sliding block switch at the end of each laser diode mount that could be moved to block laser light when the system was not in operation. The USB board camera sensors were also housed in a 3-D-printed mount, which was printed using an Anycubic 3D printer in black resin. The black resin can absorb light and reduce back reflection and stray light disturbances. Each laser diode operated by a custom-made printed circuit board combined with a laser diode driver (e.g., Thorlabs LD1100), both encased atop the laser diode.

The USB board cameras were in the form of rolling shutter cameras (e.g., Basler daA3840-45 μm with Sony IMX334 sensor). The USB board cameras operated at a framerate of 40 frame-per-second, with a pixel pitch of 2×2 μm, a pixel resolution of 3840×2160 pixels, a pixel depth of 8 bits, and operated at an exposure time τ=6 ms.

Each USB board camera included the heat management system 530 shown in FIG. 5A to dissipate heat to provide a comfortable contact temperature for the patients during extended recordings. In this implementation, the heat management system 530 included a 0.040″ laser-cut copper sheet, a 1 mm neoprene sheet (McMaster 93375K608) and an anodized aluminum heat sink of 30×20×10 mm placed on the back of the copper sheet, a 1 mm thickness cork sheet, and a 1 mm thickness black silicone sheet.

The SCOS method implemented by the SCOS system provided the GUI interface 895. The GUI interface 895 may enable non-experts to operate the portable multi-channel SCOS system 800 and display visualizations of cerebral blood metrics at the end of each recording for one or more of the channels corresponding to six locations within the brain of the subjects. Operator input may include subject information and duration of recording. FIG. 9B includes an example of CBF and CBV time trace recordings measured over 5 seconds by the six channels 841, 842, 843, 844, 845, and 846 of SCOS system 800 on a human subject. As shown, the CBF and CBV time traces exhibit the same frequency of oscillation and are temporally synchronized. It is noted that the CBF time traces contain more high frequency information than the CBV time traces, including variations such as the peak systole or dicrotic notch of the cardiac pulse. Additionally, the CBF time traces show greater temporal stability as compared to the CBV time traces, as minor fluctuations in blood oxygen concentration and other instabilities do not significantly impact the normalized speckle contrast calculations in Eqn. 1.

The six light detectors 831, 832, 833, 834, 835, and 836 were distributed along the skulls of the subjects as follows: two on each side of the forehead, one on the front and one on the back of the left hemisphere, and one on the front and one on the back of the right hemisphere. Each light detector was placed at a source-to-detector (S-D) distance from the corresponding light laser source of about 3.2±0.2 cm. In some cases, an S-D distance between 3.0 to 3.5 cm may correspond to an optimal brain sensitivity over signal-to-noise ratio. During operation, the laser diodes were positioned about 6 mm away from the skin of the subjects such that the illumination spot diameter was 5.5 mm. The total maximum illumination power used during operation was limited to 67 mW.

### E. Battery-Powered SCOS Systems

In certain implementations, SCOS systems described herein may include integrated energy sources that are batteries (e.g., a 9V battery) to power the laser sources (battery-powered laser sources) during operation. For example, the portable multi-channel SCOS system 800 in FIGS. 8A and 8B includes three power sources 881, 882, and 883 integrated into the headset 801.

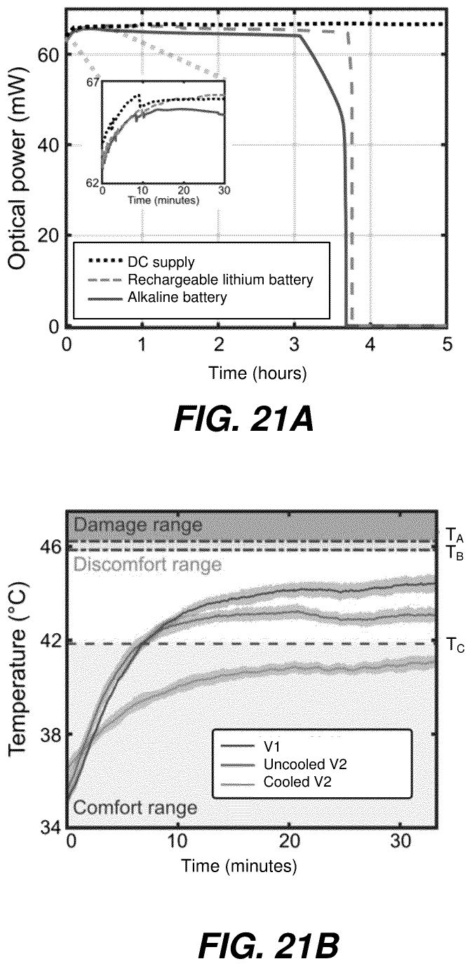

In three cases, the portable multi-channel SCOS system 800 in FIGS. 8A and 8B was employed with three different types of power sources a 9V DC bench power supply, a 9V alkaline battery, and a rechargeable 9V lithium-ion battery and the heat management system 530 in FIG. 5A. FIG. 21A is a plot of the laser optical power over time for the three different power sources a 9V DC bench power supply, a 9V alkaline battery, and a rechargeable 9V lithium-ion battery. Both the 9V alkaline battery and the rechargeable 9V lithium-ion battery had power for over 3 hours with the lithium-ion battery lasting the longest at approximately 3.7 hours. The lithium-ion battery is also the most lightweight and includes an internal electrical switch that automatically disables the battery when voltage falls below a threshold which can avoid noisy data collection during the discharge phase. With an average recording time of 15 minutes, both the 9V alkaline battery and the rechargeable 9V lithium-ion battery can support up to 12 recordings.

### E. Heat Management Systems

In certain implementations, SCOS systems may include light detector packages with heat management systems (e.g., a board camera with heat management system 530 shown in FIG. 5A).

In three cases, the portable multi-channel SCOS system 800 in FIGS. 8A and 8B was implemented with three different types of cameras: Basler camera daA1920-160 μm (V1), Basler daA3840-45 μm board camera running at 30 FPS and 8.3 million pixels (uncooled V2), Basler daA3840-45 μm board camera running at 40 FPS and 8.3 million pixels encased in the heat management system 530 in FIG. 5A (cooled V2). The contact temperature was measured by placing the system on silicone skin pads positioned on a hot plate set at 32.5° C. to simulate human skin contact. FIG. 21B is a plot of the average contact temperature measured for the three types of cameras. Based on ASTM guidelines, temperature thresholds for one hour of operation include: 1) a comfort temperature threshold T_{A}=41.85° C., 2) a reversible skin damage threshold temperature T_{B}=45.85° C., and 3) a severe (irreversible) skin damage threshold temperature T_{C}=46.22° C. As shown in FIG. 21B, the measured contact temperature for the cooled board camera remained below the comfort temperature threshold T_{A} during the recording duration. The measured contact temperatures for all three types of cameras remained below both the reversible skin damage threshold temperature T_{B} and the irreversible skin damage threshold temperature T_{C}.

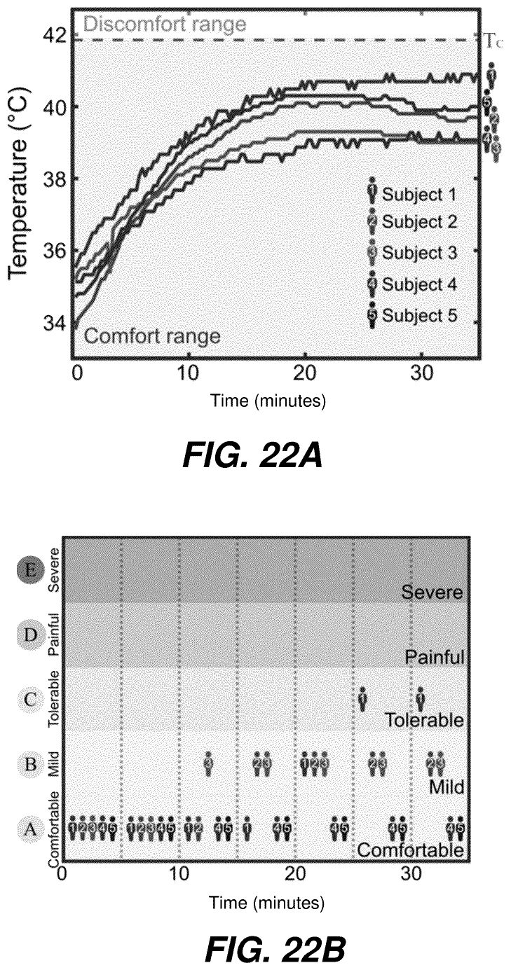

The implementation of the portable multi-channel SCOS system 800 discussed in Section V (D) above with the heat management system 530 in FIG. 5A was used to evaluate the contact temperature and comfort levels for five healthy subjects by placing the light detection package on the foreheads of the subjects during recording. The contact temperature was measured while running the board camera for a recording duration of 35 minutes with repeated 3-minute image acquisition sessions followed by 30-second breaks between image acquisitions sessions. The measurements were taken by a thermocouple positioned between the light detector package and the foreheads of the subjects. FIG. 22A is a plot of the contact temperature measurements over time taken at the contact surface of the light detector packages to the foreheads of the five healthy human subjects. As shown, the maximum temperatures reached during the recording duration were below the discomfort temperature, Tc. The subjects rated their comfort levels during the recording operation on a scale of “comfortable,” “tolerable,” “painful,” and “severe.” FIG. 22B is a plot of subject comfort ratings over time during the 35 minute recording duration provided by the five healthy human subjects. The data indicates that the implementation of the portable multi-channel SCOS system 800 can operate for at least 35 minutes without the risk of heat-induced skin damage while maintaining an adequate level of user comfort.

### F. Likelihood of Brain Injury

In certain implementations, SCOS systems may be utilized to detect and/or characterize a brain injury or other brain malfunction. The implementation of the portable multi-channel SCOS system 800 discussed in Section V(D) above was used to measure CBF and CBF of a healthy subject, a subject with traumatic brain injury (TBI), and a subject with non-traumatic brain injury (NTBI). The NTBI subject had more evidence of structural brain damage than the TBI subject. The TBI subject had a decompressive hemicraniectomy and cranioplasty skull implant.

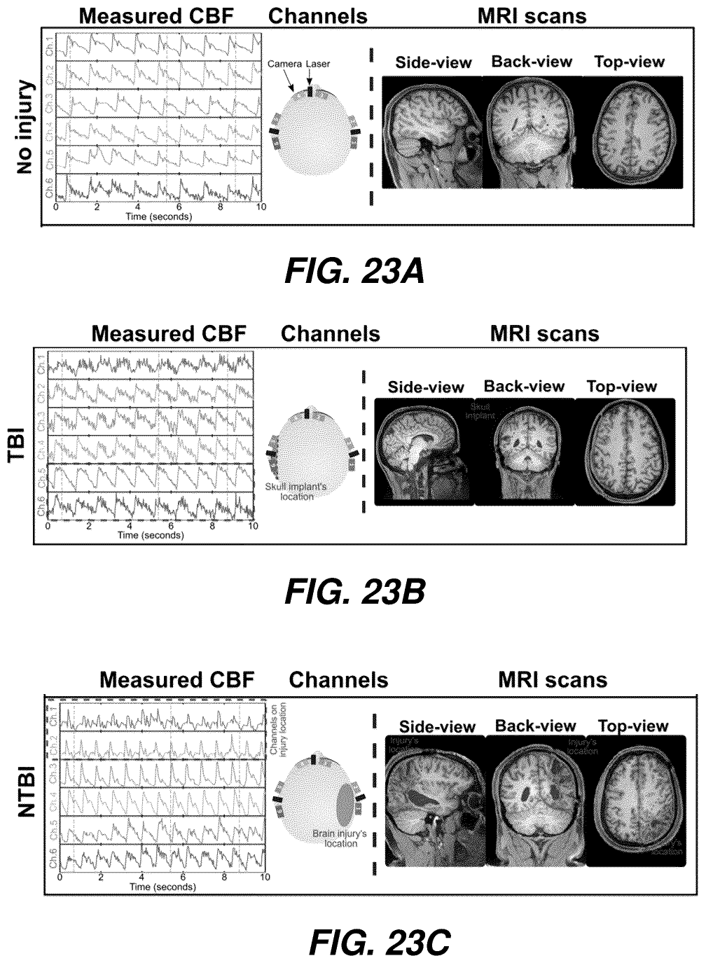

FIG. 23A includes a plot of the time traces of the measured CBF at six different regions in the brain of a healthy subject (“No injury”) and MRI scans. FIG. 23B includes a plot of time traces of the measured CBF at six different regions in the brain of a subject having traumatic brain injury (TBI), an image of the location of the skull implant, and MRI scans. FIG. 23C includes a plot of time traces of the measured CBF at six different regions in the brain of a subject having non-traumatic brain injury (NTBI), an image of the location of the brain injury, and MRI scans.

As shown in FIG. 23A, the CBF data measured for the healthy subject shows synchronized and highly correlated blood flow dynamics across all six channels corresponding to the six regions of the brain. A similar pattern is observed in the TBI subject. The MRI scans confirmed that neither the healthy subject nor the TBI subject had significant brain damage. As shown in FIG. 23B, the CBF data measured for the TBI subject exhibited synchronized and correlated blood flow dynamics across all channels including the two channels (Channel 5 and 6) positioned over the area of the skull implant's location. As shown in FIG. 23C, the CBF data measured for the NTBI subject exhibited synchronized and correlated blood flow dynamics across all channels except the two channels (Channels 1 and 2) positioned over the area of brain injury with structural brain damage revealed by the MRI scan. In these examples, the difference in blood flow dynamics between channels is indicative of structural damage.

In various embodiments, a SCOS system can determine an indication of structural damage at a region within the brain based on a two-channel correlation of blood flow metrics between two channels. A two-channel correlation factor, p, refers to a linear correlation between the cerebral blood flow metrics of two channels in a multi-channel SCOS system. A two-channel correlation factor, p, for CBF can be determined by:

$$ρ ( i , j ) = \frac{{\sum }_{t = 1}^{T} ( CBFI ( i , t ) - \overline{CBFI} ( i ) ) ( CBFI ( j , t ) - \overline{CBFI} ( j ) )}{\sqrt{{\sum }_{t = 1}^{T} {( CBFI ( i , t ) - \overline{CBFI} ( i ) )}^{2} {\sum }_{t = 1}^{T} {( CBFI ( j , t ) - \overline{CBFI} ( j ) )}^{2}}} , \qquad ( Eqn . 17 )$$

where CBFI(i,t) and CBFI(j,t) are the cerebral blood flow index at channel i and j respectively, C̅B̅F̅I̅(̅i̅)̅ and C̅B̅F̅I̅(̅j̅)̅ are the average CBFI over time, and τ is the total number of datapoints in the time trace. A two-channel correlation factor, p, for CBV can be determined similarly.

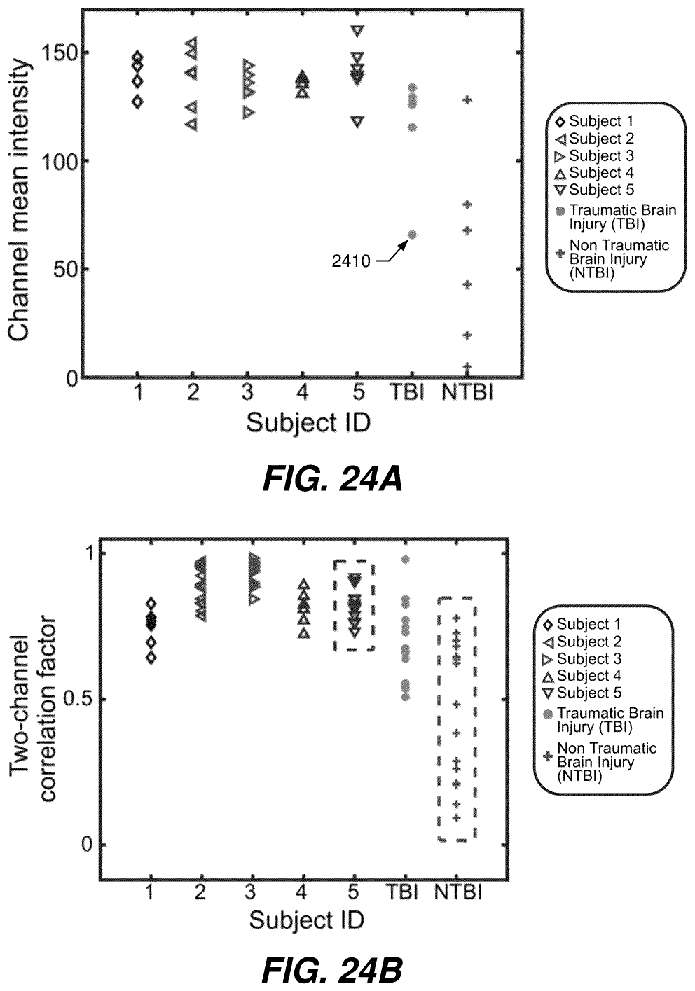

For the six channel example, the total number of two channel combinations and associated correlation factors is fifteen (15). FIG. 24A is a plot of the mean intensity of raw image recordings at six channels for the five healthy subjects, the TBI subject, and the NTBI subject. FIG. 24B is a plot of the two-channel correlation factors from CBF recordings at the six channels for the five healthy subjects, the TBI subject, and the NTBI subject.

As shown in FIG. 24B, the two-channel correlation factors show high correlation between the channels for all five healthy subjects. As shown in FIG. 24A, the mean intensity values are very similar for all channels for five healthy subjects. As shown in FIG. 24B, the TBI subject with the skull implant exhibited similar channel mean intensity to the healthy subjects except for one channel with mean intensity 2410 but exhibited lower two-channel correlation factor values between channels. The NTBI subject exhibited significantly lower mean intensity and two-channel correlation factor values, especially on the four channels located on the back-right side of the head, i.e. the channels closer to the injury location.

FIG. 25A depicts a correlation matrix with correlation factors for channel pairs of all six channels determined for a healthy subject based on the CBF time traces shown in FIG. 23A. FIG. 25B depicts a correlation matrix with correlation factors for channel pairs of all six channels determined for an NTBI subject based on the CBF time traces shown in FIG. 23C. While the correlation factor values between all six channels of the healthy subject are high (i.e., >0.75), the correlation factors between channels of the NTBI subject reveal two distinct groups. Channels 1 and 2 near the brain injury are correlated with each other as shown by grouping 2510, and Channels 3 to 6 away from the brain injury form a separate grouping 2520 that correlate with each other. The channels (Channels 1 and 2) positioned over the brain injury site are different as compared to the other channels (Channels 3-6) on the other side of the brain without brain injury. The two-channel correlation factors between Channels 1 and 2 and channels on the other side of the brain without brain injury are lower as compared with the two-channel correlation factors in groupings 2510, 2520.

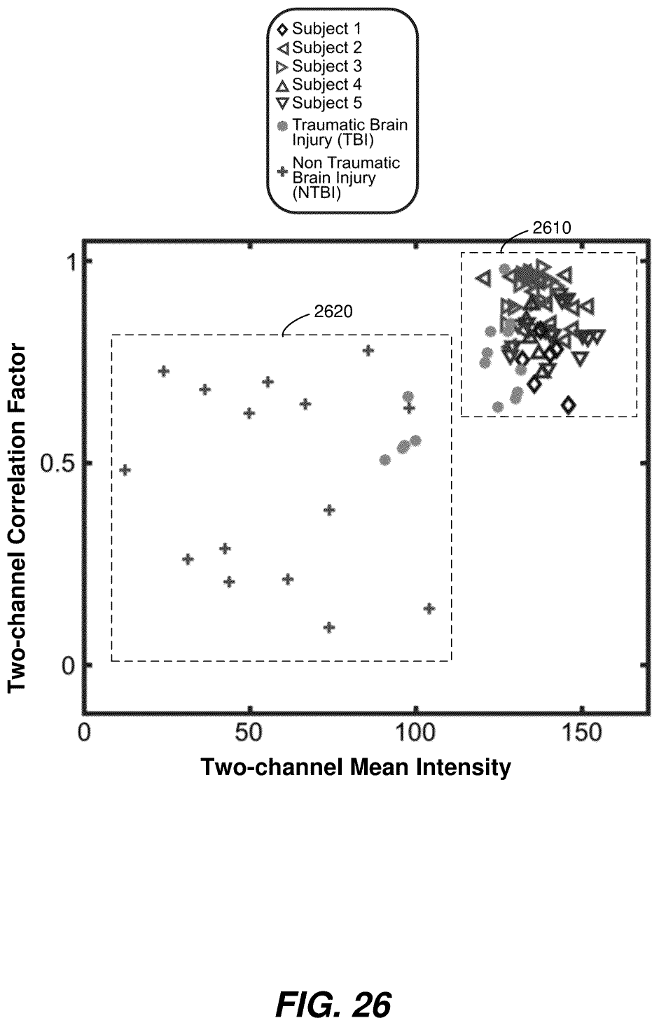

FIG. 26 is a plot of the two-channel correlation factors and two-channel mean intensities for all channels and all subjects. The two-channel mean intensity can be calculated by averaging the mean intensity of the two channels used to compute the correlation factor. The mean intensity is related to cerebral blood volume. As shown by the grouping 2610, all subjects except the NTBI subject exhibited high correlation factors and high mean intensities, indicating synchronized CBF and CBV dynamics with the same pulse waveform across all six channels for the subjects. In contrast, the NTBI subject with structural brain damage (in grouping 2620) displayed higher variability in correlation factors and mean intensities, suggesting regional differences in CBF and CBV, likely due to the channels being located proximate to the injured region exhibiting different blood flow dynamics than the other channels.

### VI. Examples of Methods for Detecting Brain Injury or Other Brain Malfunction

Certain embodiments pertain to techniques that are cost-effective, comprehensive, and sufficiently reliable for clinical use to characterize brain injuries beyond structural damage, particularly the persistent disruption of normal cerebrovascular reactivity and cerebral blood flow. These techniques implement speckle contrast optical spectroscopy (SCOS) to apply laser speckle contrast imaging to non-invasively monitor cerebral blood flow metrics in subjects. In some implementations, an infrared laser source is used to transmit light through the skull and into the brain. By transmitting infrared light through one location on the skull and collecting its transmission with a light detector (e.g., camera) on another location, the SCOS system can determine cerebral blood volume by measuring the light attenuation rate. Since the light used is coherent (laser), it is possible to also determine cerebral blood flow rate by recording how fast the transmitted speckles fluctuate.

In certain embodiments, multi-channel SCOS systems can perform simultaneous, non-invasive measurement of CBF and CBV at different brain locations (regional cerebrovascular monitoring) and detect the likelihood of a brain injury or other brain malfunction in at least one of the regions based in part on the measurements of CBF and/or CBV. In addition or alternatively, these multi-channel SCOS systems can be used to categorize brain injury as traumatic brain injury or non-traumatic brain injury. In one example, a multi-channel SCOS system includes a headset with a wearable headband and a plurality of channels disposed on the headband. Each channel may include at least one laser diode and a board camera having a light detector (e.g., Basler daA3840-45 μm camera board with Sony IMX392 CMOS sensor). In some cases, channels on a headband may share the same light detector. The multi-channel SCOS systems may also include an electrical connector (e.g., USB connection) with one end for coupling to the board camera and another end in electrical communication with a computing device. The multi-channel SCOS systems may also include an integrated power source such as a rechargeable battery.

In various embodiments, SCOS systems may be able to detect a likelihood of a brain injury or other brain malfunction based on measured blood flow metrics. For example, in certain implementations, multi-channel SCOS systems (e.g., multi-channel SCOS system 800 in FIG. 8) can detect brain injuries or other brain malfunctions based on two-channel correlation factors between channel pairs or other two channel factors related to CBF between channel pairs. A two-channel correlation factor may be determined using Eqn. 17. Another two-channel factor that can be used is a two-channel mean intensity which is the average mean intensity of the two channels. As another example, SCOS systems detect a likelihood of a brain injury or other brain malfunction by comparing morphological features of at least one first waveform of a time trace of a blood flow metric of the subject with the waveform of a time trace of a blood flow metric of a reference healthy subject.

### Detecting Brain Injury Using Two-Channel Factors

According to certain embodiments, multi-channel SCOS systems can probe different regions of the brain. The differences in CBF and/or CBV determined at the different regions can be used to determine a likelihood of brain injury or other brain malfunction.

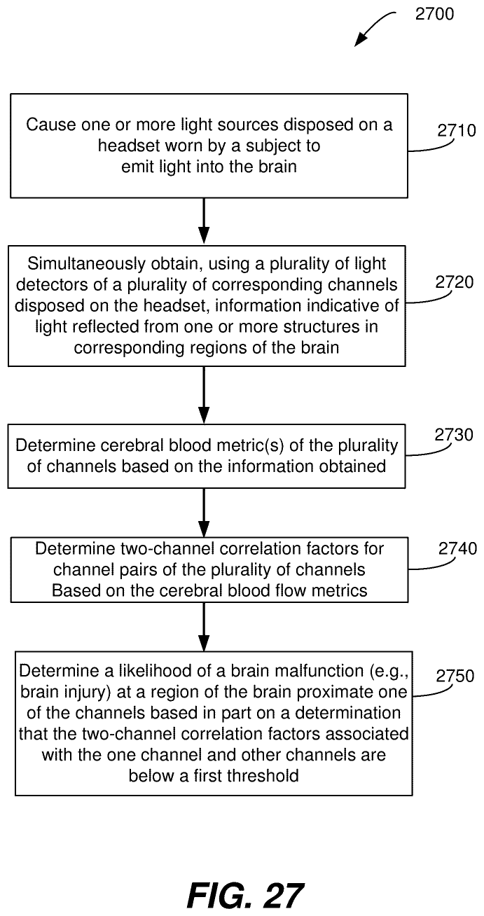

FIG. 27 is a flow diagram 2700 depicting a method of determining a likelihood of a brain malfunction such as a brain injury, according to some embodiments. Examples of multi-channel SCOS systems (e.g., multi-channel SCOS system 800 in FIG. 8, multi-channel SCOS system 1100 in FIG. 11) can be used to implement the operations. The multi-channel SCOS system includes multiple light source and light detector pairs forming respective channels to probe different regions of the brain simultaneously. Any suitable number of channels may be used (e.g., 2, 4, 6, 8, etc.). In some instances, two or more channels may share the same light source. For example, as shown in FIG. 8B, channel 1 841 shares a light source 730 with channel 2 842.

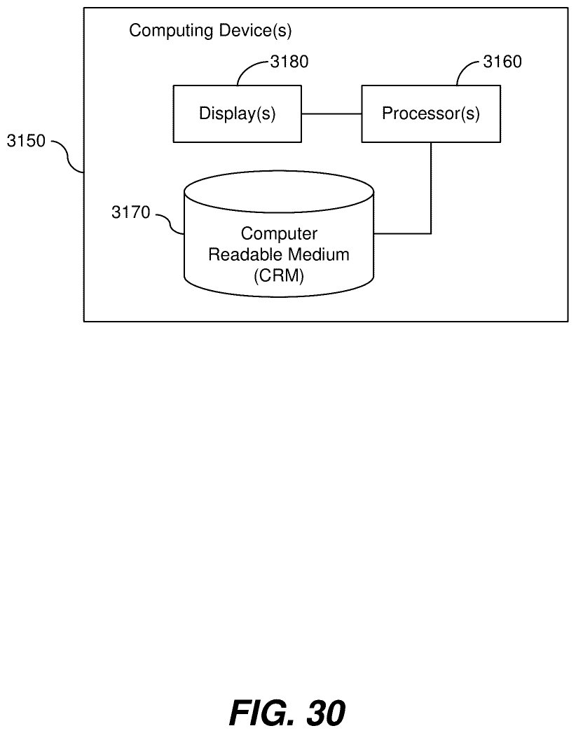

Blocks of the flow diagram 2700 may be executed by one or more processors of one or more computing devices. An example of such a computing device is shown in and described below in connection with FIGS. 8A-8C and FIG. 30. Note that, in some embodiments, at least one of the one or more computing devices may be disposed on a headband or headset on which one or more light sources and light detectors are disposed. Accordingly, in some such embodiments, cerebral blood metrics, extracted features associated with the cerebral blood metrics, and/or likelihood of a brain malfunction may be determined by a computing device itself on the headband or headset. Alternatively, in some embodiments, data obtained by the light detectors, and/or data representative of the cerebral blood metrics may be transmitted from a computing device disposed on the headband or headset to a second computing device remote from or separate from the headband or headset, where the second computing device generates the likelihood of a brain malfunction. In some implementations, the blocks of flow diagram 2700 may be executed in an order other than what is shown in FIG. 27. In some embodiments, one or more blocks of process 2700 may be omitted, and/or two or more blocks may be executed substantially in parallel.

At 2710, the method can begin by causing, using one or more light sources disposed on a headset worn by a subject (also referred to herein as a user), light to be emitted into the head of the subject. In many instances, the light from the one or more light sources is transmitted through the skull into the brain. An example of a headset is the headset 801 shown in and described above in connection with FIGS. 8A and 8B. The one or more light sources may include one or more lasers, one or more LEDs, etc. In some implementations, the one or more light sources may emit light in an infrared or near infrared wavelength region. In some implementations, multiple light sources may be disposed on a headset or headband. Note that light may be emitted continuously, or may be pulsed.

At 2720, the method simultaneously obtains information indicative of light reflected from one or more structures at different regions within the brain of the subject. The information is obtained using a plurality of light detectors of a plurality of corresponding channels. The light detectors are disposed on the headband and/or within the headset. The information is collected by the light detectors from corresponding regions of the brain probed at depths that correspond to the relative distance between the one or more light sources and the light detectors (S-D distances). Each light detector may include a camera, a photodetector, etc. In one example, light emitted by a laser may be reflected from various head and brain structures and may be captured by a camera (e.g., to determine speckle contrast, as described above). Note that, as shown in and described above in connection with FIGS. 8A and 8B, because the SCOS system includes multiple (e.g., two, four, eight, ten, etc.) channels, the obtained information corresponds to different brain regions (e.g., a left frontal lobe region, a right frontal lobe region, a left parietal lobe region, a right parietal lobe region, etc.) which are generally proximate the light detectors. In some cases, the information from each light detector and associated channel can be represented as a time trace.

At 2730, the method can, based on the obtained information, determine one or more cerebral blood metrics for the regions probed by the light detectors at the plurality of channels. In some cases, the cerebral blood metrics may include one or both of a cerebral blood volume and a cerebral blood flow. The cerebral blood volume may be determined based on the intensity of the reflected light signal at a single wavelength. The cerebral blood flow may be determined using DCS and/or SCOS.

At 2740, the method can determine a two-channel correlation factor for each of the channel pairs of the plurality of channels. For example, the SCOS system 800 in FIG. 8A has six channels with 15 channel pairs. The two-channel correlation factor can be determined using Eqn. 17. FIG. 25B depicts an example of a matrix including two-channel correlation factors for the 15 channel pairs of the six channel SCOS system 800 in FIG. 8A for a subject with a brain injury in a region proximate channels 1 and 2. In some cases, the cerebral blood metrics data for each channel can be represented as a time trace. In another implementation, another two-channel factor may be used.

At 2750, the method can determine a likelihood that, given the cerebral blood metrics data, the subject has a brain malfunction proximate one of the channels. The method determines the likelihood based on a determination that one or more two-channel correlation factors associated with the one channel is less than a first threshold (e.g., 0.60, 0.50, and 0.40). In some cases, the one or more correlation factors may be associated with the one channel and other channels that are located at positions on the skull away from the position of the one channel (e.g. on the opposite side of the head). By way of example, referring to the two-channel correlation factors in FIG. 25B, the two-channel correlation factor between channel 1 and channel 2 is 0.62, the two-channel correlation factor between channel 1 and channel 3 is 0.21, the two-channel correlation factor between channel 1 and channel 4 is 0.38, the two-channel correlation factor between channel 1 and channel 5 is 0.26, and the two-channel correlation factor between channel 1 and channel 6 is 0.48. The first threshold may be 0.50 in this example. The method may determine that the three two-channel correlation factors between channel 1 and channels 3-6 at other sides of the head from channel 1 are below the first threshold of 0.50. The method may determine that there is a high likelihood that the subject has a brain malfunction proximate channel 1 based on this determination. The method may output likelihood data to an operator of the SCOS system. The likelihood data may be a number on a discrete scale (e.g., an integer between 1 and 5), a number on a continuous scale (e.g., a probability value that is a continuous number between 0 and 1), or the like.

In one embodiment, the method may determine the likelihood that the subject has a brain malfunction based further on a determination that two-channel mean intensity values between the one channel and the other channels are below a second threshold. The two-channel mean intensity values are related to cerebral blood volume. In various cases, the second threshold may be 0.60, 0.50, or 0.40.

In one embodiment, the method may also be used to determine the likelihood that the subject does not have a brain malfunction at a region proximate the one channel based on a determination that the two-channel correlation factors determined for all channel pairs are above a third threshold. In various cases, the third threshold may be 0.70, 0.80, or 0.90. By way of example, referring to the two-channel correlation factors in FIG. 25A for a healthy subject, the two-channel correlation factor between channel 1 and channel 2 is 0.92, the two-channel correlation factor between channel 1 and channel 3 is 0.82, the two-channel correlation factor between channel 1 and channel 4 is 0.90, the two-channel correlation factor between channel 1 and channel 5 is 0.91, and the two-channel correlation factor between channel 1 and channel 6 is 0.85. The third threshold may be 0.80 in this example. The method may determine that given the two-channel correlation factors between channel 1 and all the other channels is greater than 0.80, the other channels are highly correlated with channel 1 and there is a high likelihood that the subject does not have a brain malfunction at a region proximate channel 1.

### Detecting Brain Injury Based on Morphology of Time Traces of Cerebral Blood Metrics

In some embodiments, a SCOS system can detect brain injury or other brain malfunction at a region within the brain by comparing the morphological features (e.g., peaks and dicrotic notch) of a periodic waveform of a time trace of cerebral blood metrics of the subject to morphological features of a reference time trace of a healthy subject.

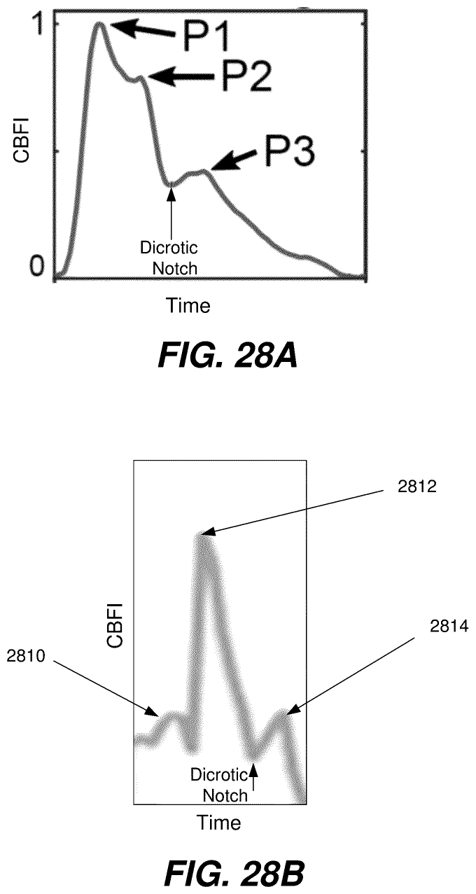

FIG. 28A is a plot of an example of one reference pressure waveform of a time trace of CBFI in a healthy subject. The reference waveform includes a (first) peak pressure (P1), a second pressure (P2) following the first peak pressure, a dicrotic notch indicating the end of systole and beginning of diastole, and a third pressure (P3). FIG. 28B is a plot of an example of a pressure waveform of a time trace of CBFI measured in the NTBI subject at channel 1 proximate the brain injury. As shown in FIG. 28B, the time trace of the of the includes peaks 2810 before the highest peak 2812 indicating a likelihood of brain malfunction.

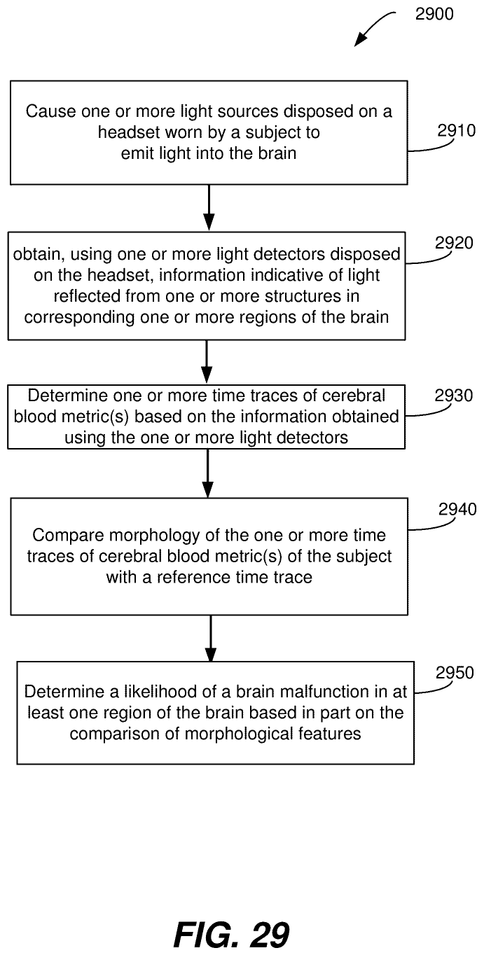

FIG. 29 is a flow diagram 2900 depicting a method of determining a likelihood of a brain malfunction such as a brain injury, according to some embodiments. Various examples of SCOS systems described herein can be used to implement the operations. The SCOS systems may be multi-channel or may have a single channel. The SCOS system implemented includes one or more light sources and one or more light detectors.

Blocks of the flow diagram 2900 may be executed by one or more processors of one or more computing devices. An example of such a computing device is shown in and described below in connection with FIGS. 8A-8C and FIG. 30. Note that, in some embodiments, at least one of the one or more computing devices may be disposed on a headband or headset on which one or more light sources and light detectors are disposed. Accordingly, in some such embodiments, cerebral blood metrics, extracted features associated with the cerebral blood metrics, and/or likelihood of a brain malfunction may be determined by a computing device itself on the headband or headset. Alternatively, in some embodiments, data obtained by the light detectors, and/or data representative of the cerebral blood metrics may be transmitted from a computing device disposed on the headband or headset to a second computing device remote from or separate from the headband or headset, where the second computing device generates the likelihood of a brain malfunction. In some implementations, the blocks of flow diagram 2900 may be executed in an order other than what is shown in FIG. 29. In some embodiments, one or more blocks of process 2900 may be omitted, and/or two or more blocks may be executed substantially in parallel.

At 2910, the method can begin by causing, using one or more light sources disposed on a headset worn by a subject (also referred to herein as a user), light to be emitted into the head of the subject. In many instances, the light from the one or more light sources is transmitted through the skull into the brain. An example of a headset is the headset 801 shown in and described above in connection with FIGS. 8A and 8B. In some implementations, the one or more light sources may emit light in an infrared or near infrared wavelength region. In some implementations, two light sources of different types (e.g., a laser and an LED), each of which may emit light in a different wavelength region (e.g., infrared and near infrared) may be packaged together as a light emission package. In some implementations, multiple light emission packages may be disposed on the headset or headband, each configured to emit light into different regions of the user's head or brain. Note that light may be emitted continuously, or may be pulsed.

At 2920, the method uses one or more light detectors to obtain information indicative of light reflected from one or more structures at one or more regions within the brain of the subject. The one or more light detectors are disposed on the headband and/or within the headset. The information is collected by the light detectors from corresponding regions of the brain probed at depths that correspond to the relative distance between the one or more light sources and the light detectors (S-D distances). Each light detector may include a camera, a photodetector, etc. In one example, light emitted by a laser may be reflected from various head and brain structures and may be captured by a camera (e.g., to determine speckle contrast, as described above). In In some implementations, the SCOS system implemented may include multiple (e.g., two, four, eight, ten, etc.) channels and the obtained information corresponds to different brain regions (e.g., a left frontal lobe region, a right frontal lobe region, a left parietal lobe region, a right parietal lobe region, etc.) which are generally proximate the corresponding light detectors.

At 2930, the method can, based on the obtained information, determine one or more time traces of cerebral blood metrics for one or more regions probed by the one or more light detectors. In some cases, the cerebral blood metrics may include one or both of a cerebral blood volume and a cerebral blood flow. The cerebral blood volume may be determined based on the intensity of the reflected light signal at a single wavelength. The cerebral blood flow may be determined using SCOS.

At 2940, the method can compare morphological features of the one or more time traces of cerebral blood metrics for the one or more regions of the brain of the subject with a reference time trace. In some cases, the reference time trace may be a measured time trace of a cerebral blood metric of a region of a brain of a healthy subject or other representation of a such a time trace.

At 2950, the method can determine a likelihood that, given the cerebral blood metrics evaluated, the subject has a brain malfunction in at least one region of the brain probed based on the comparison of morphological features. The method may output likelihood data to an operator of the SCOS system. The likelihood data may be a number on a discrete scale (e.g., an integer between 1 and 5), a number on a continuous scale (e.g., a probability value that is a continuous number between 0 and 1), or the like.

### VII. Computational Devices

The techniques described above may be implemented using one or more computing devices. FIG. 30 illustrates components of an example computing device that may be used, e.g., to implement blocks of process 1200 of FIG. 12, process 1300 of FIG. 13, process 1400 of FIG. 14, method in FIG. 27, or method in FIG. 29, respectively. Note that such a computing device may be part of a headset comprising one or more light sources and/or one or more light detectors (e.g., the computing device may be disposed on a portion of the headset or headband), or may be communicatively coupled to the headset (e.g., via a wireless communication channel, such as BLUETOOTH).

In FIG. 30, the computing device(s) 3050 includes one or more processors 3060 (e.g., microprocessors), a non-transitory computer readable medium (CRM) 3070 in communication with the processor(s) 3060, and one or more displays 3080 also in communication with processor(s) 3060. Processor(s) 3060 is in electronic communication with CRM 3070 (e.g., memory). Processor(s) 3060 is also in electronic communication with display(s) 3080, e.g., to display image data, text, etc. on display 3080. Processor(s) 3060 may retrieve and execute instructions stored on the CRM 3070 to perform one or more functions described above. For example, processor(s) 3060 may execute instructions to perform one or more operations to analyze collected data (e.g., light reflection/absorption data). The CRM (e.g., memory) 3070 can store instructions for performing one or more functions of the described above. These instructions may be executable by processor(s) 3060. CRM 3070 can also store raw images, e.g., speckle images, or the like.

### VIII. Example Embodiments

Embodiment 1: A headset for generating cerebral blood metric data, the headset comprising: a headband configured to be worn on a head; and a laser coupled to the headband and configured to emit light into a brain within a skull of the head; a light detector coupled to the headband and configured to generate information indicative of light reflected from one or more structures within the brain, wherein the light detector is configured to contact or be within 5 mm of a scalp of the skull; and a power source coupled to the headband and in electrical communication with the laser.

Embodiment 2: The headset of embodiment 1, wherein the headset is configured to be able to adjust a distance between the laser and the light detector.

Embodiment 3: The headset of embodiment 1, wherein the laser is a continuous laser.

Embodiment 4: The headset of any one of the embodiments 1-3, wherein the laser is configured to emit light in a near-infrared or infrared wavelength.

Embodiment 5: The headset of embodiment 1, wherein the headband comprises one or more straps.

Embodiment 6: The headset of embodiment 1, wherein the headband is configured to encircle the head.

Embodiment 7: The headset of embodiment 1, wherein the headband is configured to place the light detector in contact with a region of a scalp of the head.

Embodiment 8: The headset of embodiment 7, wherein the headband is further configured to apply pressure to the scalp at the region of the scalp in contact with the light detector.

Embodiment 9: The headset of any one of embodiments 1-8, further comprising one or more processors configured to: cause, using the laser, light to be emitted into the brain; obtain, using the light detector, the information indicative of the reflected light from one more structures within the brain; and based on the obtained information, determine one or more cerebral blood metrics.

Embodiment 10: The headset of embodiment 9, wherein the one or more cerebral blood metrics comprises one or more of a cerebral blood flow, a cerebral blood volume, or a heart rate.

Embodiment 11: The headset of embodiment 9, wherein the one or more cerebral blood metrics comprises one or more of a cerebral blood flow, a cerebral blood volume, or a heart rate in a region of the brain.

Embodiment 12: The headset of embodiment 1, wherein the light detector is configured to probe a region of the brain.

Embodiment 13: The headset of embodiment 1, wherein the laser, the light detector, and the power source form a first channel.

Embodiment 14: The headset of embodiment 13, further comprising one or more additional channels, wherein each additional channel comprises an additional light detector and wherein the additional light detectors of the additional channels are positioned to probe different regions of the brain.

Embodiment 15: The headset of embodiment 14, wherein at least one of the additional channels comprises the laser and the power source of the first channel.

Embodiment 16: The headset of embodiment 1, wherein the light detector comprises a plurality of sensor segments.

Embodiment 17: The headset of embodiment 16, wherein the sensor segments are configured to probe different regions at different depths of the brain.

Embodiment 18: The headset of embodiment 17, wherein each of the sensor segments comprises a complementary metal-oxide-semiconductor sensor.

Embodiment 19: A multi-channel headset comprising: a headband configured to be worn on a head during operation; and a plurality of channels coupled to the headband, each channel comprising: a laser configured to emit light into a brain within a skull of the head; a light detector configured to generate information indicative of light emitted by the laser and reflected by one or more structures within the brain; and a power source in electrical communication with the laser; wherein the light detectors of the channels are configured to probe different regions of the brain.

Embodiment 20: The multi-channel headset of embodiment 19, wherein at least two of the channels have light detectors configured to receive light from the same laser.

Embodiment 21: An system comprising: a circuit board; one or more processors attached to the circuit board; a laser diode attached to the circuit board; a light detector in electrical communication with the one or more processors; a light block located between the laser diode and the light detector; and a power source in electrical communication with the laser diode.

Embodiment 22: The system of embodiment 21, further comprising a wireless receiver for transmitting wireless signals, the wireless receiver in electrical communication with the one or more processors.

Embodiment 23: The system of embodiment 21, wherein the light block has a height in a range of 3 mm and 1 cm and a length in a range of 2 cm and 5 cm.

Embodiment 24: The system of any one of embodiments 21-23, wherein the one or more processors comprise one or both of an application-specific integrated circuit and a programmable logic device.

Embodiment 25: The system of embodiment 24, wherein the programmable logic device is a field-programmable gate array.

Embodiment 26: The system of any of embodiment 21-25, wherein the circuit board and/or the light detector is made of a flexible material.

Embodiment 27: The system of embodiment 21, wherein the light detector has a length in a range of 15 mm and 25 mm.

Embodiment 28: The system of embodiment 21, wherein the light detector is positioned such that its length is perpendicular to a surface of the light block.

Embodiment 29: The system of any one of embodiments 21-28, wherein the light detector comprises a plurality of complementary metal-oxide-semiconductor sensors.

Embodiment 30: The system of any one of embodiments 21-29, wherein the light detector comprises a plurality of sensor segments.

Embodiment 31: The system of embodiment 30, wherein the sensor segments are configured to probe different regions at different depths.

Embodiment 32: The system of any one of embodiments 21-32, wherein the one or more processors are configured to: cause, using the laser diode, light to be emitted into a brain within a skull of a head of a user; obtain, using the light detector, information indicative of light reflected from one more structures within the brain; and based on the obtained information, determine one or more cerebral blood metrics.

Embodiment 33: The system of embodiment 32, wherein the obtained information comprises a speckle pattern obtained using images captured by the light detector.

Embodiment 34: The system of embodiment 32, wherein a portion of the obtained information spans a time period during which the user was holding their breath.

Embodiment 35: The system of embodiment 33, wherein the one or more cerebral blood metrics comprise a cerebral blood flow determined based on the speckle pattern.

Embodiment 36: The system of any one of embodiments 21-35, further comprising a headband configured for attachment to a head, wherein the circuit board is attached to the headband such that the light detector is configured to contact or be in close proximity to a scalp of the head.

Embodiment 37: The system of embodiment 36, further comprising one or more additional circuit boards attached to the headband, each circuit board comprising an additional laser diode, an additional light detector, an additional light block, and an additional power source.

Embodiment 38: A method of determining one or more cerebral blood metrics, the method comprising: causing, using a laser, light to be emitted into a brain within a skull of a head of a user; obtaining, using a light detector, information indicative of light reflected from one more structures within the brain; and based on the obtained information, determining one or more cerebral blood metrics as a function of time.

Embodiment 39: The method of embodiment 38, wherein the laser and the light detector are disposed on a headset worn by the user.

Embodiment 40: The method of embodiment 38, wherein the obtained information comprises a plurality of speckle images.

Embodiment 41: The method of embodiment 40, further comprising: normalizing each speckle image based on a first set of the speckle images acquired during a first time period; calculating a speckle contrast of each normalized speckle image; and adjusting the speckle contrast to account for noise.

Embodiment 42: The method of any one of embodiments 40-41, further comprising calculating a cerebral blood flow from the adjusted speckle contrast of each image of a second set of the speckle images acquired during a second time period in which the user was holding their breath.

Embodiment 43: The method of any one of embodiments 40-42, further comprising: calculating a plurality of cerebral blood flow values over time from the adjusted speckle contrast of a second set of the speckle images acquired during a second time period in which the user was holding their breath; and calculating a heart rate from a period in the cerebral blood flow values over time.

Embodiment 44: A multi-channel speckle contrast optical spectroscopy system comprising: a headset with a headband configured to encircle a head of a subject; one or more light sources attached to the headband; a plurality of light detectors attached to the headband, the plurality of light detectors corresponding to a plurality of channels; and one or more processors configured to: cause, using one or more light sources disposed on a headset worn by a subject, light to be emitted into a brain of the subject; simultaneously obtain, using light detectors of a plurality of channels disposed on the headset, information indicative of light reflected from one more structures in a plurality of corresponding regions within the brain; based on the obtained information, determine one or more cerebral blood metrics for the plurality of channels; determine two-channel correlation factors for channel pairs of the plurality of channels based on the one or more cerebral blood metrics determined; and determine a likelihood of brain malfunction at a region proximate one of the channels based on part on a determination that one or more of the two-channel correlation factors associated with the one of the channels is less than a first threshold.

Embodiment 45: The multi-channel headset of embodiment 44, wherein the one or more processors are configured to predict the brain malfunction at the first region of the brain further based on a determination that a two-channel mean intensity between the first channel and the other one of the channels is below a second threshold.

Embodiment 46: The multi-channel headset of embodiment 44, wherein the at least two light sources comprise a laser configured to emit light in an infrared wavelength range.

Embodiment 47: The multi-channel headset of embodiment 44, wherein the one or more light detectors comprise a camera.

Embodiment 48: The multi-channel headset of embodiment 44, further comprising a heat management system for extracting heat from the plurality of light detectors to an ambient environment.

Embodiment 49: The multi-channel headset of embodiment 44, further comprising a heat management system for extracting heat from the plurality of light detectors to an ambient environment.

Embodiment 50: The multi-channel headset of embodiment 44, wherein the plurality of light detectors comprises a segmented image sensor.

Embodiment 51: A speckle contrast optical spectroscopy method comprising: causing, using one or more light sources disposed on a headset worn by a subject, light to be emitted into a brain of the subject; simultaneously obtaining, using light detectors of a plurality of channels disposed on the headset, information indicative of light reflected from one more structures in a plurality of corresponding regions within the brain; based on the obtained information, determining one or more cerebral blood metrics for the plurality of channels; determining two-channel correlation factors for channel pairs of the plurality of channels based on the one or more cerebral blood metrics determined; and determining the likelihood of brain malfunction at a region proximate one of the channels based on part on a determination that one or more of the two-channel correlation factors associated with the one of the channels is less than a first threshold.

Embodiment 52: The method of embodiment 51, wherein the first threshold is about 0.60, about 0.50, or about 0.40.

Embodiment 53: The method of embodiment 51, wherein the prediction of the brain malfunction at the first region of the brain is further based on a determination that two-channel mean intensity values between the one of the channels and the other channels is below a second threshold.

Embodiment 54: The method of embodiment 53, wherein the second threshold is about 0.60, about 0.50, or about 0.40.

Embodiment 55: The method of embodiment 51, further comprising predicting a likelihood of no brain malfunction based in part on a determination that the two-channel correlation factors determined for all the channel pairs are above a third threshold.

Embodiment 56: The method of embodiment 55, wherein the third threshold is about 0.70, about 0.80, or about 0.90.

Embodiment 57: The method of embodiment 51, wherein the brain malfunction is a brain injury.

Embodiment 58: The method of embodiment 51, wherein the brain malfunction is a traumatic or a non-traumatic brain injury.

Embodiment 59: The method of embodiment 51, wherein the one or more cerebral blood metrics comprise at least one of cerebral blood flow and cerebral blood volume.

Embodiment 60: The method of embodiment 51, wherein the one or more cerebral blood metrics comprise a cerebral blood flow determined based on a decorrelation time associated with a series of speckle patterns obtained from a series of images captured by the one or more light detectors.

Embodiment 61: A speckle contrast optical spectroscopy method comprising: causing, using one or more light sources disposed on a headset worn by a subject, light to be emitted into a brain of the subject; obtaining, using one or more light detectors disposed on the headset, information indicative of light reflected from one more structures in a plurality of corresponding regions within the brain; based on the obtained information, determining one or more time traces of a cerebral blood metric at one or more corresponding regions of the brain; comparing morphological features of the one or more time traces with a reference time trace; and predicting the brain malfunction at a region of the brain proximal one or more light detectors based in part on the comparison.

Embodiment 62: The method of embodiment 61, wherein the morphological features comprise one or more peaks and a dicrotic notch.

Embodiment 62: The method of embodiment 61, wherein the reference time trace is of a healthy subject.

Embodiment 62: The method of embodiment 61, wherein a brain malfunction is predicted if the morphological features include one or more peaks and a dicrotic notch.

Embodiment 62: The method of embodiment 61, wherein the brain malfunction is a brain injury.

Embodiment 62: The method of embodiment 61, wherein the brain malfunction is a traumatic or a non-traumatic brain injury.

Embodiment 62: The method of embodiment 61, wherein the one or more cerebral blood metrics comprise at least one of cerebral blood flow and cerebral blood volume.

Embodiment 62: The method of embodiment 61, wherein the one or more cerebral blood metrics comprise a cerebral blood flow determined based on a decorrelation time associated with a series of speckle patterns obtained from a series of images captured by the one or more light detectors.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C #, C++ or Python, Matlab, or other suitable language/computational software, including low level code, including code written for field programmable gate arrays, for example in VHDL; embedded artificial intelligence computing platform, for example in Jetson. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random-access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. A multi-channel speckle contrast optical spectroscopy system, comprising:
a headset with a headband configured to encircle a head of a subject;
one or more light sources attached to the headband;
a plurality of light detectors attached to the headband, the plurality of light detectors corresponding to a plurality of channels; and
one or more processors configured to:
cause, using one or more light sources disposed on a headset worn by a subject, light to be emitted into a brain of the subject;
simultaneously obtain, using light detectors of a plurality of channels disposed on the headset, information indicative of light reflected from one more structures in a plurality of corresponding regions within the brain;
based on the obtained information, determine one or more cerebral blood metrics for the plurality of channels;
determine two-channel correlation factors for channel pairs of the plurality of channels based on the one or more cerebral blood metrics determined; and
determine a likelihood of brain malfunction at a region proximate one of the channels based in part on a determination that one or more of the two-channel correlation factors associated with the one of the channels is less than a first threshold.

2. The multi-channel speckle contrast optical spectroscopy system of claim 1, wherein the one or more processors are configured to determine the likelihood of the brain malfunction at the region of the brain further based on a determination that a two-channel mean intensity between the one of the channels and other channels is less than a second threshold.

3. The multi-channel speckle contrast optical spectroscopy system of claim 1, wherein the one or more processors are further configured to predict low brain malfunction or no brain malfunction based in part on a determination that all two-channel correlation factors determined for the channel pairs of the plurality of channels are greater than a third threshold.

4. The multi-channel speckle contrast optical spectroscopy system of claim 1 wherein the one or more light sources comprise a laser configured to emit light in an infrared wavelength range.

5. The multi-channel speckle contrast optical spectroscopy system of claim 1, wherein the one or more light detectors comprise a camera.

6. The multi-channel speckle contrast optical spectroscopy system of claim 1, further comprising a heat management system for extracting heat from the plurality of light detectors to an ambient environment.

7. The multi-channel speckle contrast optical spectroscopy system of claim 1, wherein the plurality of light detectors comprises a segmented image sensor.

8. A speckle contrast optical spectroscopy method, comprising:
causing, using one or more light sources disposed on a headset worn by a subject, light to be emitted into a brain of the subject;
simultaneously obtaining, using light detectors of a plurality of channels disposed on the headset, information indicative of light reflected from one more structures in a plurality of corresponding regions within the brain;
based on the obtained information, determining one or more cerebral blood metrics for the plurality of channels;
determining two-channel correlation factors for channel pairs of the plurality of channels based on the one or more cerebral blood metrics determined; and
determining a likelihood of brain malfunction at a region proximate one of the channels based on part on a determination that one or more of the two-channel correlation factors of the channel pairs including the one of the channels is less than a first threshold.

9. The speckle contrast optical spectroscopy method of claim 8, wherein the first threshold is about 0.60, about 0.50, or about 0.40.

10. The speckle contrast optical spectroscopy method of claim 8, wherein the determination of the brain malfunction at the first of the brain is further based on a determination that two-channel mean intensity values of the channel pairs including the one of the channels is below a second threshold.

11. The speckle contrast optical spectroscopy method of claim 10, wherein the second threshold is about 0.60, about 0.50, or about 0.40.

12. The speckle contrast optical spectroscopy method of claim 8, further comprising predicting a likelihood of no brain malfunction based in part on a determination that the two-channel correlation factors determined for all the channel pairs are above a third threshold.

13. The speckle contrast optical spectroscopy method of claim 12, wherein the third threshold is about 0.70, about 0.80, or about 0.90.

14. The speckle contrast optical spectroscopy method of claim 8, wherein the brain malfunction is a traumatic or a non-traumatic brain injury.

15. The speckle contrast optical spectroscopy method of claim 8, wherein the one or more cerebral blood metrics comprise at least one of cerebral blood flow and cerebral blood volume.

16. The speckle contrast optical spectroscopy method of claim 8, wherein the one or more cerebral blood metrics comprise a cerebral blood flow determined based on a decorrelation time associated with a series of speckle patterns obtained from a series of images captured by the light detectors.

17. A speckle contrast optical spectroscopy method, the method comprising:
causing, using one or more light sources disposed on a headset worn by a subject, light to be emitted into a brain of the subject;
obtaining, using one or more light detectors disposed on the headset, information indicative of light reflected from one more structures in one or more corresponding regions within the brain;
based on the obtained information, determining one or more time traces of a cerebral blood metric at the one or more corresponding regions of the brain;
comparing morphological features of the one or more time traces with morphological features of a reference time trace; and
predicting the brain malfunction at least one of the one or more regions based in part on the comparison of morphological features.

18. The speckle contrast optical spectroscopy method of claim 17, wherein the morphological features being compared comprise a peak and a dicrotic notch.

19. The speckle contrast optical spectroscopy method of claim 17, wherein the reference time trace is of a healthy subject.

20. The speckle contrast optical spectroscopy method of claim 17, wherein the brain malfunction is a traumatic brain injury or a non-traumatic brain injury.
